(19) United States
(12) Patent Application Publication
(10) Pub. No.: US 2023/0139146 A1
(43) Pub. Date: May 4, 2023

(54) DYEING OR LIGHTENING PROCESS USING A HAND-HELD STYLING DEVICE AND A SUBSTRATE

(71) Applicant: L'OREAL, Paris (FR)

(72) Inventors: Leila HERCOUET, Saint Ouen (FR); Simon DONCK, Saint Ouen (FR); Henri SAMAIN, Chevilly-Larue (FR)

(73) Assignee: L'OREAL, Paris (FR)

(21) Appl. No.: 17/778,356

(22) PCT Filed: Nov. 19, 2020
(86) PCT No.: PCT/EP2020/082763

§ 371 Date: May 19, 2022

(30) Foreign Application Priority Data
Nov. 20, 2019 (FR) FR1912967

(51) Int. Cl. A45D 19/00; A61K 8/22; A61K 8/02; A61Q 5/06; A61Q 5/08

(52) U.S. Cl. CPC A45D 19/0066; A45D 19/012; A61K 8/22; A61K 8/0208; A61Q 5/065; A61Q 5/08; A61K 2800/4322; A61K 2800/87

(57) ABSTRACT

The present invention relates to the use, for dyeing or lightening keratin fibres, in particular human keratin fibres such as the hair, of a hand-held styling apparatus (1) equipped with at least one treatment surface (2d, 3d) partially or totally covered with at least one substrate (6) comprising a surface (6a) coated with at least one layer (6b) containing one or more dyes, preferably chosen from oxidation dyes and/or direct dyes, and/or one or more oxidizing agents. The invention also relates to an assembly (11, 24, 26) intended for dyeing or lightening keratin fibres, in particular human keratin fibres such as the hair, comprising at least one aqueous composition and at least one hand-held styling device (1) as defined above.

The present invention relates to the field of dyeing or lightening keratin fibres and more particularly to the field of dyeing and lightening the hair.

The present invention relates in particular to a process for dyeing or lightening keratin fibres, in particular human keratin fibres such as the hair, comprising the use on said fibres of at least one aqueous composition and of the hand-held styling device as defined below, which has at least one treatment surface partially or totally covered with at least one substrate comprising a surface coated with at least one layer containing one or more dyes and/or oxidizing agents. Preferably, the dyes are chosen from oxidation dyes and/or direct dyes which are synthetic or natural, more preferably oxidation dyes.

The invention also relates to an assembly intended for dyeing or lightening keratin fibres, in particular human keratin fibres such as the hair, comprising at least one aqueous composition and at least one hand-held styling device as defined below, and preferably at least one computing device configured for calculating the number of uses of the substrate on said fibres and at least one image acquisition device configured for visualizing the surface of the substrate put in place on said keratin fibres.

Standard oxidation dyeing processes generally consist in applying to keratin fibres a dyeing composition comprising oxidation bases and couplers with hydrogen peroxide (H₂O₂ or aqueous hydrogen peroxide solution) as oxidizing agent, in leaving it to diffuse, and then in rinsing said fibres. The colourings resulting therefrom are generally permanent, strong and resistant to external agents, notably to light, bad weather, washing, perspiration and rubbing.

The “semi-permanent” dyeing or direct dyeing processes, for their part, consist in applying, to said keratin fibres, direct dyes, which are coloured and colouring molecules that have affinity for said fibres, in leaving them on for a time, and then in rinsing them off. In order to perform these dyeing operations, the direct dyes generally used are chosen from nitrobenzene, anthraquinone, nitropyridine, azo, xanthene, acridine, azine and triarylmethane direct dyes, and natural dyes.

In addition, in order to vary the shades obtained in oxidation dyeing, or to enrich them with tints, it is also known practice to use oxidation dyes in combination with direct dyes.

However, it has been found that it is often difficult or even virtually impossible to obtain colourings with novel visual results by means of “standard” dyeing techniques. In particular, such processes do not satisfactorily afford coloured or multi-coloured patterns, which may lead to novel, appealing optical effects on the entire head of hair, which prove to be both sharp and precise.

These standard dyeing processes also have the drawback of staining the hands of the user or of the hair stylist during the application to the hair of the dyeing composition based on direct dyes or of the ready-to-use composition resulting from mixing the dyeing composition and the oxidizing composition. Similarly, this type of process may also give rise to undesirable stains on the user's scalp, contour of the face and/or clothing, which may be due to application errors and/or to problems of running of the compositions.

In particular, the standard oxidation dyeing processes also entail the risk of not leading to the final colouring desired by the user because of an error arising during the handling of the dyeing compositions and oxidizing compositions or because of a poor choice of the starting dyeing compositions.

It has also been found that the storage of the dyeing compositions and/or oxidizing compositions used for obtaining the desired colouring may pose problems of bulkiness, notably in hairstyling salons.

These standard dyeing processes may thus prove to be impractical for achieving many varied colours as a function of the different users.

In order to overcome these various drawbacks, it has already been proposed in FR 3 015 895 to perform a dyeing process consisting in placing the keratin fibres in contact with a substrate which is in the form of a sheet, which has been pretreated with a composition comprising one or more oxidation dyes, and then with an aqueous oxidizing composition. By means of this pretreatment, the oxidation dyes form a layer which partly or totally coats the surface of the substrate. The oxidation dyes may thus be deposited onto the surface of the substrate uniformly or non-uniformly and may adopt one or more novel geometric shapes in order to produce different types of coloured patterns.

In particular, the keratin fibres are placed on the substrate and the oxidizing composition is then applied to said fibres and/or the substrate. In the course of the dyeing process, the oxidation dyes are extracted from the surface of the substrate so as to migrate towards the keratin fibres to penetrate them and will react with the oxidizing agent(s) of the oxidizing composition so as to dye the fibres.

Such a process has the advantage of leading to persistent colourings which may be uniform, multi-coloured and/or bearing coloured patterns making it possible to produce, for example, shading-off, spots, novel geometric shapes and/or reproductions of images on the keratin fibres. In particular, such a process makes it possible to create visually novel coloured patterns that can be personalized by the consumer.

A lightening process which brings the keratin fibres into contact with a substrate in the form of a sheet similar to the dyeing process has also been proposed in document FR 3 015 892.

The use of such substrates makes it possible to reduce the problems of bulkiness linked to the storage of the dyeing compositions and oxidizing compositions used in standard dyeing processes. This is because the substrates take up less space than the dyeing or lightening compositions generally used. Thus, the process gives the user the possibility of creating a wide range of coloured or bleached patterns while at the same time benefiting from more space in terms of storage in hair salons.

However, such a process involves leave-on times that prove to be quite long, generally of the order of 30 minutes, and which are in particular due to the fact that the oxidation dyes or the oxidizing agents must first be extracted from the substrate before dyeing or lightening the keratin fibres.

In order to shorten this leave-on time, it has been envisaged to modify the very nature of the substrate so as to promote faster extraction of the dyes so that they can react with the oxidizing agent(s) of the oxidizing composition and access the keratin fibres more easily.

To this end, it has already been proposed to use substrates which do not absorb dyes, notably using plasticized sheets.

However, the leave-on times observed with substrates of this type still too often remain long, namely generally of the order of 25 minutes.

Furthermore, this type of substrate is problematic as regards large-scale production. Specifically, during the pretreatment of the substrate with the composition containing the oxidation dye(s) or the oxidizing agent(s), said composition may be incorrectly deposited and may produce pools on the surface of the substrate, which has a negative impact on the sharpness of the coloured patterns.

Moreover, it is been found that the use of the substrates described in document FR 3 015 895 or FR 3 015 892 can still prove to be impractical, in particular when it is a question of producing extremely varied coloured or bleached patterns at various places on the head of hair, since this can involve regularly changing the substrates as a function of the areas treated. As a result of this, the dyeing or lightening process can be perceived as being long and painstaking by both the stylist and the consumer.

Similarly, these problems in terms of use can also be encountered when the stylist wants to use such substrates on different models (i.e. different heads of hair) in order to create varied coloured or bleached patterns.

The disadvantages described above are also encountered in the context of a dyeing process using substrates which have been pretreated by means of direct dyes and also in the context of a lightening process using substrates which have been pretreated with oxidizing agents.

There is therefore a real need to overcome the drawbacks mentioned above, in particular by carrying out a process capable of simply and rapidly producing sharp and precise coloured or bleached patterns on keratin fibres, which poses minimal problems in terms of use while at the same time reducing the problems of bulkiness and also the risks of contact that can occur between the compositions used and the hands, the scalp and/or the clothing of the user.

This aim is achieved by the present invention, one subject of which is in particular a process for dyeing and/or lightening keratin fibres, in particular human keratin fibres such as the hair, using, on said fibres, at least one aqueous composition and at least one hand-held styling device comprising:
a first and a second jaw arranged facing one another and connected to one another so as to go from an open position to a closed position,
a first treatment surface, borne by the first jaw, and a second treatment surface, borne by the second jaw, the surfaces being intended to grip said keratin fibres, and at least one of the surfaces being intended to be heated,
at least one of the treatment surfaces being partially or totally covered with at least one substrate comprising a surface coated with at least one layer containing one or more dyes, preferably chosen from oxidation dyes and/or direct dyes, even more preferably chosen from oxidation dyes, and/or one or more chemical oxidizing agents.

The dyeing or lightening process according to the invention has the advantage of producing coloured or bleached patterns with considerably shorter leave-on times, thereby making it particularly effective compared to the standard dyeing or lightening processes.

The dyeing or lightening process according to the invention also makes it possible to produce on the keratin fibres, with great precision, coloured or bleached patterns that are visually sharp. More particularly, the process makes it possible to produce millimetre-sized coloured or bleached patterns having all types of shapes, such as spots or waves, which are readily reproducible. These patterns may also lead to novel optical effects when they are then repeated over the entire head of hair.

In other words, the dyeing or lightening process according to the invention makes it possible to obtain patterns, especially millimetre-sized patterns, homogeneously over the entire head of hair, or in a localized manner on a part of the head of hair. These patterns may be imaginative from an aesthetic viewpoint or may serve to hide an irregularity in the colour or appearance of the keratin fibres, especially in the case of hair regrowth or fading of the ends.

Thus, the process makes it possible to result in a dyeing and/or lightening of keratin fibres by producing coloured or bleached patterns.

In addition, the dyeing or lightening process according to the invention proves to be easier to carry out when it is the aim to produce identical or varied coloured or bleached patterns on the entire head of hair than a process in which the substrate is not used in combination with a hand-held styling device.

Indeed, the hand-held styling device used in accordance with the present invention makes it possible to effectively apply one or more identical or different substrates simply and rapidly to keratin fibres and to produce sharp and precise coloured or bleached patterns which give said fibres an original and attractive aesthetic appearance.

In particular, the substrates intended to be applied to the keratin fibres can be rapidly installed in and/or removed from the hand-held styling device during the dyeing or lightening process.

Furthermore, the hand-held styling device makes it possible to preserve the advantage of using substrates which can be easily stored in hair salons and which can be prepared on site or in advance.

The dyeing process according to the invention also makes it possible to obtain colourings and/or patterns of which the colourings are powerful and persistent with respect to external agents (such as shampooing, light, perspiration or bad weather).

In particular, the dyeing process according to the invention leads to the production of patterns of which the colouring is powerful and resistant with respect to shampooing.

Furthermore, the colour build-up brought about by the process according to the invention is high.

The present invention also relates to an assembly intended for dyeing or lightening keratin fibres, in particular human keratin fibres such as the hair, comprising at least one aqueous composition and at least one hand-held styling device as defined above.

The assembly according to the invention is intended to be used during the process for dyeing or lightening keratin fibres, in particular human keratin fibres such as the hair, as defined above.

The assembly makes it possible to rapidly and effectively produce coloured or bleached patterns in short treatment times.

Preferably, the assembly according to the invention can comprise:

i) an aqueous composition as defined below,

ii) a hand-held styling device as defined above,

iii) a computing device configured for calculating the number of uses of the substrate(s) on the keratin fibres,
the hand-held styling device also comprising one or more means for detecting the moving into an open position and/or the moving into a closed position of the first and the second jaw; said one or more detection means being connected to the computing device.

More preferably, the assembly according to the invention also comprises an image acquisition device, configured for visualizing on a screen the surface of the substrate(s) intended to be used on the keratin fibres; the image acquisition device being connected to the computing device.

The assembly according to the invention is intended to be used during a process for dyeing or lightening keratin fibres, in particular human keratin fibres such as the hair.

In particular, the assembly according to the invention makes it possible to visualise and/or automate all or part of the production of coloured or bleached patterns on the keratin fibres and also to control the type of patterns produced (i.e. their geometric shape and/or their colour) and their number.

The assembly according to the invention also makes it possible to calculate the number of coloured or bleached patterns that remain to be produced on the keratin fibres.

Thus, the assembly according to the invention makes it possible to effectively assist the user of a hand-held styling device as described above in order to produce colourings or lightenings, in particular coloured or bleached patterns, in short times.

Other subjects, characteristics, aspects and advantages of the invention will emerge even more clearly on reading the description and the examples that follow.

For the purposes of the present invention, and unless otherwise indicated:
a “heteroaryl radical” represents an optionally cationic, 5- to 22-membered monocyclic or fused or non-fused polycyclic group, comprising from 1 to 6 heteroatoms chosen from nitrogen, oxygen, sulfur and selenium, and at least one ring of which is aromatic; preferably, a heteroaryl radical is chosen from acridinyl, benzimidazolyl, benzobistriazolyl, benzopyrazolyl, benzopyridazinyl, benzoquinolyl, benzothiazolyl, benzotriazolyl, benzoxazolyl, pyridyl, tetrazolyl, dihydrothiazolyl, imidazopyridyl, imidazolyl, indolyl, isoquinolyl, naphthoimidazolyl, naphthooxazolyl, naphthopyrazolyl, oxadiazolyl, oxazolyl, oxazolopyridyl, phenazinyl, phenoxazolyl, pyrazinyl, pyrazolyl, pyrilyl, pyrazoyltriazyl, pyridyl, pyridinoimidazolyl, pyrrolyl, quinolyl, tetrazolyl, thiadiazolyl, thiazolyl, thiazolopyridyl, thiazoylimidazolyl, thiopyrylyl, triazolyl, xanthyl and thioxanthinyl;
an “aryl” radical represents a monocyclic or fused or non-fused polycyclic carbon-based group, comprising from 6 to 22 carbon atoms, at least one ring of which is aromatic; preferably, the aryl radical is a phenyl, biphenyl, naphthyl, indenyl, anthracenyl or tetrahydronaphthyl;
the “aryl” or “heteroaryl” radicals or the aryl or heteroaryl part of a radical may be substituted with at least one substituent borne by a carbon atom, chosen from:
a C₁-C₆ and preferably C₁-C₄ alkyl radical optionally substituted with one or more radicals chosen from hydroxyl, C₁-C₂ alkoxy, (poly)hydroxy(C₂-C₄)alkoxy, acylamino, amino substituted with two C₁-C₄ alkyl radicals, which may be identical or different, optionally bearing at least one hydroxyl group, or the two radicals possibly forming, with the nitrogen atom to which they are attached, a saturated or unsaturated, optionally substituted 5- to 7-membered and preferably 5- or 6-membered heterocycle optionally comprising another nitrogen or non-nitrogen heteroatom;
a halogen atom;
a hydroxyl or thiol group;
a C₁-C₆ alkoxy or C₁-C₆ alkylthio radical;
a (poly)hydroxy(C₂-C₆)alkoxy radical;
an amino radical;
a 5- or 6-membered heterocycloalkyl radical, preferably morpholino, piperazino, piperidino or pyrrolidino, which is optionally substituted with a (C₁-C₄)alkyl radical, preferably methyl;
a 5- or 6-membered heteroaryl radical, preferably imidazolyl, optionally substituted with a (C₁-C₄)alkyl radical, preferably methyl;
an amino radical substituted with one or two identical or different C₁-C₆ alkyl radicals, optionally bearing at least:
a hydroxyl group,
an amino group optionally substituted with one or two optionally substituted C₁-C₃ alkyl radicals, said alkyl radicals possibly forming with the nitrogen atom to which they are attached a saturated or unsaturated, optionally substituted 5- to 7-membered heterocycle, optionally comprising at least one other nitrogen or non-nitrogen heteroatom,
a quaternary ammonium group —N⁺R′R″R′″, M⁻ for which R′, R″ and R′″, which may be identical or different, represent a hydrogen atom or a C₁-C₄ alkyl group and M⁻ represents an anionic counterion,
or an optionally cationic 5- or 6-membered heteroaryl radical, preferably imidazolium, optionally substituted with a (C₁-C₄)alkyl radical, preferably methyl;
an acylamino radical (—N(R)—C(O)—R′) in which the radical R is a hydrogen atom or a C₁-C₄ alkyl radical optionally bearing at least one hydroxyl group and the radical R′ is a C₁-C₂ alkyl radical;
a carbamoyl radical ((R)₂N—C(O)—) in which the radicals R, which may be identical or different, represent a hydrogen atom or a C₁-C₄ alkyl radical optionally bearing at least one hydroxyl group;
an alkylsulfonylamino radical (R′—S(O)₂—N(R)′) in which the radical R represents a hydrogen atom or a C₁-C₄ alkyl radical optionally bearing at least one hydroxyl group and the radical R′ represents a C₁-C₄ alkyl radical, or a phenyl radical;
an aminosulfonyl radical ((R)₂N—S(O)₂—) in which the radicals R, which may be identical or different, represent a hydrogen atom or a C₁-C₄ alkyl radical optionally bearing at least one hydroxyl group;
a carboxylic radical in acid or salified (preferably with an alkali metal or a substituted or unsubstituted ammonium) form;
a cyano group;
a nitro or nitroso group;
a polyhaloalkyl group, preferably trifluoromethyl;
a cyclic or heterocyclic radical, or a non-aromatic part of an aryl or heteroaryl radical, may also be substituted with one or more oxo groups;
an “alkyl radical” is a linear or branched C₁-C₁₀, in particular C₁-C₈, more particularly C₁-C₆ and preferably C₁-C₄ hydrocarbon-based radical.

The limits of a range of values are included in that range, notably in the expressions “between . . . and . . . ” and “ranging from . . . to . . . ”. The expression “at least one” is equivalent to the expression “one or more” and may be replaced therewith.

Dyeing and/or Lightening Process

Dyeing and/or Lightening Process Steps

As indicated above, the present invention relates to a process for dyeing and/or lightening keratin fibres, in particular human keratin fibres such as the hair, using, on said fibres, at least one aqueous composition, preferably a composition comprising one or more oxidizing agents, and at least one hand-held styling device as defined above.

Preferably, the process for dyeing and/or lightening keratin fibres comprises:
using an aqueous composition, as defined below, on said fibres,
bringing the treatment surfaces of the hand-held styling device, as defined above, into contact with, preferably applying them to, said keratin fibres,
increasing the temperature of said fibres to a temperature ranging from 60 to 250° C.

Preferably, the process for dyeing and/or lightening keratin fibres comprises the application of an aqueous composition to the keratin fibres.

During the implementation of the dyeing and/or lightening process, the keratin fibres are thus conveyed between the treatment surfaces of the hand-held styling device.

The treatment surfaces grip the keratin fibres while moving from an open position to a closed position and at least one of the treatment surfaces intended to be heated increase(s) the temperature of the fibres to a temperature ranging from 60 to 250° C., preferably to a temperature ranging from 80 to 180° C., preferably ranging from 100° C. to 160° C. and better still from 120 to 150° C.

The keratin fibres are thus brought into contact with the substrate(s) covering at least one of the treatment surfaces.

The increasing of the temperature of the keratin fibres to a temperature ranging from 60 to 250° C. occurs during the contacting of said fibres with the treatment surfaces of the hand-held styling device.

Preferably, the heating step is performed for a time which may range from 1 to 30 seconds and preferably from 1 to 10 seconds.

At the end of the time for leaving the substrate(s) on the keratin fibres, the treatment surfaces go from a closed position to an open position.

Advantageously, the dyeing or the lightening of the keratin fibres can be obtained after a few seconds.

At the end of the use of the hand-held styling device, the keratin fibres can be rinsed or washed with a shampoo.

Preferably, the dyeing or lightening process successively uses on keratin fibres, in particular human keratin fibres such as the hair, at least one aqueous composition as defined above, preferably a composition comprising one or more chemical oxidizing agents, then the hand-held styling device as defined above.

Preferably, the aqueous composition comprises one or more chemical oxidizing agents.

Preferably, the substrate comprises a surface coated with at least one layer containing one or more oxidation dyes.

The aqueous composition is rinsed off or not, preferably not.

Preferably, the process comprises a rinsing step between the application of the aqueous composition and the use of the hand-held styling device.

In particular, the process comprises a step of rinsing of the keratin fibres at the end of the leave-on time of composition.

The aqueous composition may be used on the keratin fibres at ambient temperature, in particular at a temperature which may range from 23° C. to 33° C.

The composition may be applied to the keratin fibres for a leave-on time which may range from 30 seconds to 2 hours.

The aqueous composition may be applied using an applicator, in particular a brush, or by hand.

At the end of the leave-on time of the composition, the keratin fibres may be rinsed with water.

Preferably, the hand-held styling device is used on said keratin fibres less than 3 hours after the application of the composition, preferably less than 1 hour, better still less than 45 minutes, after the application of the composition.

In particular, the use of the styling device is carried out less than 3 hours after the end of the leave-on time of the aqueous composition, preferably less than 1 hour, better still less than 45 minutes after the end of the leave-on time of the aqueous composition.

As a variant, the dyeing and/or lightening process successively uses on keratin fibres, in particular human keratin fibres such as the hair, the hand-held styling device as defined above and at least one aqueous composition, preferably a composition comprising one or more chemical oxidizing agents, then the hand-held styling device as defined above.

Aqueous Composition

The composition may comprise one or more chemical oxidizing agents.

The expression “chemical oxidizing agent” is intended to mean any chemical oxidizing agent other than atmospheric oxygen.

Preferably, the chemical oxidizing agents are chosen from hydrogen peroxide, urea peroxide, alkali metal bromates or ferricyanides, peroxygenated salts, for instance alkali metal or alkaline-earth metal persulfates, perborates and percarbonates, and also peracids and precursors thereof.

According to one preferred embodiment, the chemical oxidizing agents are chosen from hydrogen peroxide and/or peroxygenated salts.

The peroxygenated salt(s) are notably chosen from persulfates, perborates, peracids and/or salts thereof, percarbonates, in particular of alkali metals or alkaline-earth metals, and mixtures thereof.

Preferably, the chemical oxidizing agents are chosen from peroxygenated salts, in particular persulfates.

More preferably, the peroxygenated salt(s) are chosen from sodium, potassium and ammonium persulfates, and mixtures thereof, in particular sodium persulfate.

The chemical oxidizing agent(s) may be present in a content ranging from 0.5% to 30% by weight, preferably in a content ranging from 1% to 15% by weight, relative to the total weight of the composition.

The composition may also comprise one or more alkaline agents.

The alkaline agents may be chosen from carbonates, alkanolamines such as monoethanolamine, diethanolamine, triethanolamine and also derivatives thereof, oxyethylenated and/or oxypropylenated ethylenediamines, mineral or organic hydroxides, alkali metal silicates such as sodium metasilicate, amino acids, preferably basic amino acids such as arginine, lysine, ornithine, citrulline and histidine, and also the compounds of formula (I) below:

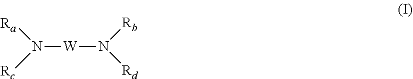

in which:
W is a divalent (C₁-C₈)alkylene group, preferably propylene, optionally substituted notably with a hydroxyl group or a C₁-C₄ alkyl radical;
Rₐ, R_{b}, R_{c} and R_{d}, which may be identical or different, represent a hydrogen atom or a C₁-C₄ alkyl or C₁-C₄ hydroxyalkyl radical.

The mineral or organic hydroxides are preferably chosen from i) hydroxides of an alkali metal, ii) hydroxides of an alkaline-earth metal, for instance sodium hydroxide or potassium hydroxide, iii) hydroxides of a transition metal, such as hydroxides of metals from groups III, IV, V and VI, iv) hydroxides of lanthanides or actinides, quaternary ammonium hydroxides and guanidinium hydroxide.

The hydroxide may be formed in situ, for instance guanidine hydroxide, by reacting calcium hydroxide with guanidine carbonate.

Preferably, the composition comprises one or more alkaline agents chosen from alkali metal silicates, in particular sodium metasilicate.

The composition may also comprise one or more surfactants, preferably one or more non-ionic and/or anionic surfactants.

Preferably, the non-ionic surfactants are oxyalkylenated and chosen from oxyethylenated C₈-C₃₀ alcohols, and polyoxyethylenated esters of saturated or unsaturated, linear or branched C₈-C₃₀ acids and of sorbitol.

The anionic surfactants may be chosen from sulfate, sulfonate, carboxylic (or carboxylate) surfactants, and mixtures thereof, notably sulfate and carboxylic surfactants.

Preferably, the anionic surfactant(s) are chosen from:
C₆-C₂₄ and notably C₁₂-C₂₀ alkyl sulfates;
C₆-C₂₄ and notably C₁₂-C₂₀ alkyl ether sulfates; preferably comprising from 2 to 20 ethylene oxide units;
C₆-C₂₄ and notably C₁₂-C₂₀ alkyl sulfosuccinates, notably lauryl sulfosuccinates;
C₆-C₂₄ and notably C₁₂-C₂₀ alkyl ether sulfosuccinates;
(C₆-C₂₄)acylisethionates, preferably (C₁₂-C₁₈)acysethionates;
C₆-C₂₄ and notably C₁₂-C₂₀ acylsarcosinates; notably palmitoyl sarcosinates;
(C₆-C₂₄)alkyl ether carboxylates, preferably (C₁₂-C₂₀)alkyl ether carboxylates;
polyoxyalkylenated (C₆-C₂₄)alkyl(amido) ether carboxylic acids and salts thereof, in particular those including from 2 to 50 alkylene oxide and in particular ethylene oxide groups;
C₆-C₂₄ and notably C₁₂-C₂₀ acylglutamates;
C₆-C₂₄ and notably C₁₂-C₂₀ acylglycinates;
   and mixtures thereof;
in particular in the form of alkali metal or alkaline-earth metal, ammonium or amino alcohol salts.

When the anionic surfactant is in salt form, said salt may be chosen from alkali metal salts, such as sodium or potassium salt, ammonium salts, amine salts and in particular amino alcohol salts, and alkaline-earth metal salts, such as magnesium salt.

Examples of amino alcohol salts that may be mentioned include monoethanolamine, diethanolamine and triethanolamine salts, monoisopropanolamine, diisopropanolamine or triisopropanolamine salts, 2-amino-2-methyl-1-propanol salts, 2-amino-2-methyl-1,3-propanediol salts and tris(hydroxymethyl)aminomethane salts.

Alkali metal or alkaline-earth metal salts and in particular sodium or magnesium salts are preferably used.

Preferably, the surfactant(s) may represent a content ranging from 0.1% to 50% by weight and preferably from 0.5% to 30% by weight relative to the total weight of the composition.

According to one embodiment, the dyeing process comprises the use on the keratin fibres of at least one aqueous composition comprising one or more chemical oxidizing agents and of a hand-held styling device as defined above and the treatment surface of which is coated with at least one layer containing one or more dyes chosen from oxidation dyes and/or direct dyes, preferably oxidation dyes.

In accordance with this embodiment, the chemical oxidizing agent(s) are preferably chosen from hydrogen peroxide and/or peroxygenated salts.

In accordance with this embodiment, the aqueous composition also preferably comprises one or more alkaline agents.

In others words, the aqueous composition is different from the substrate.

Hand-Held Styling Device

The hand-held styling device comprises:
a first and a second jaw arranged facing one another and connected to one another so as to go from an open position to a closed position,
a first treatment surface, borne by the first jaw, and a second treatment surface, borne by the second jaw, the treatment surfaces being intended to grip said keratin fibres, and at least one of the treatment surfaces being intended to be heated,
at least one of the treatment surfaces being partially or totally covered with at least one substrate comprising a surface coated with at least one layer containing one or more dyes, preferably chosen from oxidation dyes and/or direct dyes, more preferably oxidation dyes, and/or one or more chemical oxidizing agents.

For the purposes of the present invention, the term “surface intended to be heated” is intended to mean a surface of the hand-held styling device capable of increasing the temperature of the keratin fibres, in particular to a temperature ranging from 60° C. to 250° C., preferably to a temperature ranging from 80 to 180° C., more preferably to a temperature ranging from 100° C. to 160° C. and better still from 120 to 150° C.

For the purposes of the present invention, the term “treatment surface” is intended to mean a surface borne by the jaws of the hand-held styling device intended to be in contact, directly or indirectly by interposition of the substrate, with the keratin fibres for the purpose of treating them.

Preferably, the substrate(s) totally or partially cover at least one of the treatment surfaces intended to be heated.

Preferably, the two treatment surfaces of the hand-held styling device are intended to be heated.

Preferably, the two treatment surfaces of the hand-held styling device are partially or totally covered with at least two identical or different substrates.

Preferably, the substrate(s) totally cover the treatment surface(s) of the hand-held styling device. In particular, the substrate(s) partially cover the two jaws but totally cover the treatment surface(s) of the hand-held styling device.

According to one preferred embodiment, the two treatment surfaces of the hand-held styling device are totally covered with at least two identical or different substrates; at least one of said surfaces being intended to be heated; preferably, the two surfaces are intended to be heated.

According to another preferred embodiment, the hand-held styling device comprises a single treatment surface intended to be heated and is totally or partially, in particular totally, covered with at least one substrate.

In the latter case, the non-heated treatment surface is able to bring the keratin fibres into contact with said heating surface.

Preferably, the substrate(s) is or are mounted mobile on at least one of the treatment surfaces of the hand-held styling device.

Preferably, the substrate(s) is or are mounted mobile on at least one of the treatment surfaces intended to be heated of the hand-held styling device.

In other words, the substrate(s) is or are removably attached to at least one of the treatment surfaces of the hand-held styling device, preferably to at least one of the treatment surfaces intended to be heated of the hand-held styling device.

Thus, the substrate(s) is or are capable of being removed from and/or installed on at least one of the treatment surfaces of the hand-held styling device, preferably to at least one of the treatment surfaces intended to be heated of the hand-held styling device.

In this way, a substrate can be rapidly removed from the treatment surface of the hand-held styling device after production of one or more coloured or bleached patterns at a place on the head of hair, then an identical or different substrate can be installed on the same treatment surface in order to produce identical or different patterns at another place on the head of hair.

Thus, the substrate(s) can be rapidly installed on and/or removed from the hand-held styling device as a function of the type and number of patterns that it is the aim to produce.

The mobility of the substrate(s) on the treatment surface(s) of the styling device also makes it possible to renew the dyes and/or the oxidizing agents employed for producing the coloured or bleached pattern(s). This is because, at each application, the dyes are extracted from the substrate and, at the end of a certain number of repeated applications, the substrate no longer contains a sufficient amount of dyes to effectively produce precise and/or sharp coloured or bleached patterns. Thus, the removable nature of the substrate(s) on at least one of the treatment surfaces makes it possible to overcome this drawback and to anticipate any depletion of the substrate(s).

Furthermore, the mobile nature makes it possible to minimize the risks of damage, or even of destruction, of the substrate, linked to the heat of one of the treatment surfaces of the hand-held styling device.

Preferably, the substrate(s) can be mounted mobile and can be attached by means of a magnetic or mechanical, in particular mechanical, attachment system.

As a variant, the substrate(s) can be mounted mobile about at least one of the treatment surfaces of the hand-held styling device.

Preferably, the substrate(s) can be mounted mobile, totally or partially, in particular totally, about at least one of the treatment surfaces of the hand-held styling device, in particular the treatment surface intended to be heated, and can be attached with or without a magnetic attachment system.

In other words, the substrate(s) is or are capable of totally or partially, in particular totally, surrounding at least one of the treatment surfaces of the hand-held styling device with or without a magnetic attachment system.

In accordance with this embodiment, the substrate(s) totally or partially, preferably totally, cover(s) on both sides the treatment surface(s) of the hand-held styling device, preferably the treatment surface intended to be heated.

Preferably, the substrate(s) is or are oblong in shape and cover(s) on both sides at least one of the treatment surfaces of the hand-held styling device.

Preferably again, the substrate(s) form(s) a part of cylindrical shape capable of totally or partially, in particular totally, covering, in an adjusted manner, on both sides, at least one of the treatment surfaces of the hand-held styling device, in particular the treatment surface intended to be heated.

More preferably, the substrate(s) form(s) a sleeve and is or are capable of totally or partially, in particular totally, covering, on both sides, at least one of the treatment surfaces of the hand-held styling device, in particular the treatment surface intended to be heated.

According to one preferred embodiment, the two treatment surfaces of the hand-held styling device are totally or partially, preferably totally, covered on both sides with two identical or different substrates.

As a variant, the substrate(s) is or are mounted, totally or partially, in particular totally, on at least one of the treatment surfaces of the styling device, in particular the treatment surface intended to be heated, by means of a mechanical attachment system.

In accordance with this variant, the substrate(s) is or are preferably mounted, totally or partially, in particular totally, with one or more mechanical attachment means, on at least one of the treatment surfaces of the styling device, in particular on the treatment surface intended to be heated.

Preferably, the mechanical attachment means is or are clips or grippers.

According to another preferred embodiment, the substrate(s) can be mounted mobile on at least one treatment surface of the styling device, in particular the treatment surface intended to be heated, by means of at least one dispensing means which pivots about a pivoting axis, positioned on the first or second jaw, in accordance with a vertical axis Y-Y′, and is capable of being driven manually, mechanically, in a motorized manner or following a jaw closure movement.

The dispensing means is or are preferably mounted on the first or second jaw, in accordance with a vertical axis Y-Y′, and is or are intended to unwind the substrate before or after the use of the styling device, in particular after one or more applications of the substrate to the keratin fibres.

In particular, the dispensing means comprises (comprise) a rotary feed drum which has a substantially circular profile, in particular a spool, and on which the substrate is wound.

When the dispensing means is made to rotate, then the substrate unwinds from the rotary feed drum and moves onto the treatment surface of the styling device, in particular onto the treatment surface intended to be heated.

The dispensing means thus make(s) it possible to dispense with the regular operations of installing and removing the substrate(s) when it is the aim to produce coloured or bleached patterns at several places on the head of hair and on several models in reasonable times. Indeed, after each repeated application or applications of the substrate, the user can actuate the dispensing means so as to unwind the substrate and to move it to another identical or different substrate or to an as yet unused layer (or pattern) of the substrate.

The dispensing means is or are particularly advantageous for producing one or more dyeing or lightening patterns different from that or those previously produced.

The one or more dispensing means can be actuated before or after each application so as to protect the substrate from the heat of the treatment surface intended to be heated.

Preferably, the dispensing means is a reel or a barrel.

Preferably, the substrate(s) can be mounted on at least one treatment surface of the styling device, in particular the treatment surface intended to be heated, by means of two dispensing means.

According to one embodiment, the first and/or the second jaw of the hand-held styling device comprises (or comprise) one or more means for detecting the moving into the open position and/or the moving into the closed position of the jaws and connected to a computing device configured for calculating the number of applications of the substrate(s) to the keratin fibres.

The detection means make it possible in particular to indicate to the user the number of times that one and the same substrate has been used to produce one or more coloured or bleached patterns and/or the number of times that one and the same substrate can still be used before total extraction of the dyes and/or of the chemical oxidizing agents.

The computing device can be connected to an image acquisition device configured for visualizing on a screen the surface of the substrate(s) used on the keratin fibres.

The computing device can also comprise a control member intended to decrease the heating temperature of at least one of the treatment surfaces of the hand-held styling device.

Preferably, the detection means are therefore also connected to the member for controlling the computing device.

In this way, the member for controlling the computing device can cut the heating temperature of the treatment surface intended to be heated after application of the substrate to the keratin fibres. The detection means connected to the control member make it possible to protect the integrity of the dyes against the heat and to improve the lifetime of the substrates.

According to one preferred embodiment, at least one dispensing means as described above, preferably the reel, can be connected to the computing device.

Preferably, the rotary feed drum of at least one dispensing means is connected to the computing device.

Even more preferably, at least one dispensing means as described above can be connected to the computing device.

Preferably, the first and/or the second jaw of the hand-held styling device comprises (or comprise) one or more means for closing the jaws.

According to one preferred embodiment, at least one dispensing means as described above, preferably the reel, can be connected to the computing device and the first and/or the second jaw of the hand-held styling device comprises (or comprise) one or more means for closing the jaws.

Preferably, the closing means is a sensor that can be used for example when the substrate has been applied to the keratin fibres before actuating the dispensing means, preferably the reel, making it possible to place another substrate.

In this case, the system used is semi-automatic.

Preferably, the first and/or the second jaw of the hand-held styling device comprises (or comprise) one or more means for automatic closing and opening of the jaws.

More preferably, the one or more means for automatic closing and opening of the jaws include an electromagnet which ensures closing of the jaws and a return spring which ensures opening of the jaws.

According to one preferred embodiment, at least one dispensing means as described above, preferably the reel, can be connected to the computing device and the first and/or the second jaw of the hand-held styling device comprises (or comprise) one or more means for automatic closing and opening of the jaws.

In accordance with the present invention, the first and the second jaw are connected to one another in the vicinity of their end by a hinge. The two jaws can go from an open or closed position.

Preferably, the hand-held styling device is an iron, in particular a straightening iron, a curling iron, a crimping iron or else an embossing iron for keratin fibres, or an infrared ray dispenser.

Preferably, the hand-held styling apparatus is an iron, and more preferably a straightening iron.

Substrate

As indicated above, at least one of the treatment surfaces of the hand-held styling device is partially or totally covered with at least one substrate comprising a surface coated with at least one layer containing one or more dyes chosen from oxidation dyes and/or direct dyes and/or one or more chemical oxidizing agents.

Preferably, the substrate is an element in sheet form.

The element in sheet form may be made of plastic material, in particular thermoplastic, paper, a metal, especially aluminium, a woven, a nonwoven of non-absorbent fibres, especially of cellulose or a derivative thereof, or polyamide 6,6.

Preferably, the element in sheet form is a sheet of plastic material, especially of thermoplastic, or of nonwoven material of non-absorbent fibres, especially a nonwoven based on cellulose or a derivative thereof.

In particular, the element in sheet form is a sheet of nonwoven material of non-absorbent fibres, notably a nonwoven based on cellulose or a derivative thereof.

More particularly, the element in sheet form may be a paper of kraft type, which has the advantage of printing well and of leading to precise patterns. Indeed, the coloured or bleached patterns obtained on the keratin fibres do not run after the element in sheet form has been brought into contact with a fluid composition, for example with an oxidizing composition or a composition comprising a mixture based on water and/or organic solvents, or with keratin fibres that have been made wet following a prior rinsing step.

In particular, the substrate placed on the treatment surface of the hand-held styling device is an element in sheet form and is preferably a plastic sheet.

In this case, the element in sheet form has the advantage of satisfactorily rendering the colouring power, which makes it possible to lead to patterns of which the colouring is powerful. Furthermore, the plastic sheet has the advantage of not absorbing the water possibly present in an oxidizing composition or a composition based on water and/or organic solvents or possibly present following a prior step of rinsing the keratin fibres.

The element in sheet form may be constituted of a water-soluble material, which makes it possible, for example, to remove it by washing the hair.

Preferably, the element in sheet form comprises an assembly of a layer of a water-soluble material and a layer of a non-water-soluble material, for example an aluminium foil.

Preferably, the element in sheet form has a basis weight ranging from 20 to 300 g/m² and even more preferably ranging from 30 to 200 g/m².

The element in sheet form especially has a thickness ranging from 40 to 1000 micrometres, preferably a thickness ranging from 40 to 400 micrometres and better still from 60 to 200 micrometres.

The element in sheet form may be opaque or transparent. Preferably, the element in sheet form is transparent, which facilitates its positioning on the hair, especially when it is the aim to produce one or more patterns at a precise place on the lock or on the head of hair. In other words, the transparency of the element in sheet form facilitates the implementation of the dyeing or lightening process, in particular in the production of coloured or bleached patterns, and improves its precision.

The element in sheet form used in combination with the hand-held styling device is preferably flexible and strong. Preferably, the strength of the sheet is greater than 300 kPa (standard TAPPI-T403).

Preferably, the element in sheet form is water-resistant. In particular, the water absorption of said element is measured by the COBB 60 test which corresponds to the capacity of said element to absorb water during contact for 60 seconds (the procedure of which is given by standard ISO 535, TAPPI-T411 measurement).

Thus, the element in sheet form absorbs less than 100 g/m² and preferably less than 40 g/m² of water.

Preferably, the element in sheet form is resistant to the oily compounds. Thus, use may be made of a “food” paper, i.e. a complex of paper and of polymeric compound of the polyethylene type or of paper and paraffin, which is capable of acting as a barrier to water and to oils.

The element in sheet form may optionally be covered with a deposit of an adhesive composition. This adhesive layer makes it possible to improve the adhesion of the dye(s) to the surface of the element in sheet form.

According to a preferred embodiment, the element in sheet form including a surface coated with at least one layer comprising one or more dyes and/or one or more chemical oxidizing agents may be covered with a protection means which serves to protect the surface of said element from external elements. Thus, the element in sheet form comprises on its surface one or more dyes and/or one or more chemical oxidizing agents that may be covered with a protective layer. Such a protective layer makes it possible to even further minimize the impairment of the dye(s), in particular of the oxidation dyes, caused by humidity, light or atmospheric oxygen.

Thus, the element in sheet form may be protected by implementing processes used in paper varnishing techniques (oil varnish, acrylic varnish, etc.), and in particular by using a water-based or organic acrylic varnish composition.

In this way, the surface of the element in sheet form can be protected with a layer of acrylic varnish.

In other words, the element in sheet form comprises on its surface at least one layer containing one or more dyes, preferably oxidation dyes, and/or one or more chemical oxidizing agents and a layer of acrylic varnish; the layer of acrylic varnish being juxtaposed on the layer containing one or more oxidation dyes and/or one or more chemical oxidizing agents.

The mass per unit area of the layer of acrylic varnish ranges from 1 to 10 g/m² and more particularly from 2 to 5 g/m².

According to one variant, the element in sheet form is covered with a detachable protective sheet. To do this, the edges of the element in sheet form and of the protective sheet are bonded together by means of a fastening means, especially an adhesive, which may be produced via any type of method, especially by heat sealing. Thus, good cohesion is ensured between the protective sheet and the element in sheet form.

Advantageously, the protective sheet is UV-opaque to ensure better protection.

According to another variant, the element in sheet form may be covered with another protective means, namely a hermetic wrapping, defining above the element an oxygen-free space (under vacuum or under an inert atmosphere).

According to an advantageous embodiment, the element in sheet form is a plastic sheet covered with a thin layer of paper, in particular with a thickness of less than 50 μm and more preferably less than 30 μm, such as cigarette paper or a layer of paper that can be broken down in the presence of water, such as toilet paper, or a thin layer of hydrophilic material such as cellulose or a hydrophilic silica, preferably having a thickness ranging from 5 to 200 μm.

In accordance with this embodiment, the layer of thin paper allows rapid drying and prevents the colouring from running following the application of an aqueous composition as defined below. Furthermore, the layer of paper located below the thin paper does not absorb or absorbs little the dye(s) derived from the element in sheet form, as a result of its low thickness. The colouring is thus satisfactorily rendered by the layer of thin paper on the keratin fibres, which notably leads to sharp coloured or bleached patterns. Furthermore, the element in sheet form in accordance with this embodiment makes it possible to minimize the dry areas under the keratin fibres.

When use is made of a support formed from a layer of paper, which is preferably sparingly absorbent or non-absorbent, covered with a layer of paper that is capable of degrading on contact with water:

the layer of degradable paper (thickness able to range from 10 to 200 μm) allows rapid drying and prevents the colouring from running following the application of the aqueous composition as defined below. Furthermore, the layer of paper located below the degradable paper does not absorb or absorbs little of the dye(s) derived from the element in sheet form, as a result of its low thickness. The colouring is thus satisfactorily rendered by the layer of thin paper on the keratin fibres, which notably leads to sharp coloured or bleached patterns. Furthermore, the element in sheet form in accordance with this embodiment makes it possible to minimize the dry areas under the keratin fibres.

In the case where a support formed from a layer of hydrophilic material is used:

the layer of hydrophilic material is typically from 5 to 200 μm thick, which allows rapid drying and prevents the colouring from running following the application of the oxidizing aqueous composition. This results in particular in sharp coloured or bleached patterns.

According to a second advantageous embodiment, the element in sheet form is a microalveolar sheet, i.e. a sheet perforated with holes that are spaced apart from each other by a plastic material. Thus, the aqueous composition as defined below becomes housed in the holes of the substrate, which will make it possible to better render the power of the dyes on the keratin fibres after being brought into contact with the aqueous composition as defined below.

The holes are found at the surface of the element in sheet form over a thickness ranging from 10% to 90% of the thickness of the sheet.

The dye(s) is or are chosen from oxidation dyes and/or direct dyes, preferably oxidation dyes, in particular chosen from couplers and oxidation bases.

The oxidation dyes may be chosen from one or more couplers, optionally in combination with one or more oxidation bases.

Preferably, the couplers are chosen from meta-phenylenediamines, meta-aminophenols, meta-diphenols, naphthalene-based couplers and heterocyclic couplers, and also the addition salts thereof and/or the solvates thereof.

Examples that may be mentioned include 1,3-dihydroxybenzene, 1,3-dihydroxy-2-methylbenzene, 4-chloro-1,3-dihydroxybenzene, 1-hydroxy-3-aminobenzene, 1-methyl-2-hydroxy-4-β-hydroxyethylaminobenzene, 4-amino-2-hydroxytoluene, 5-amino-6-chloro-2-methylphenol, 2,4-diamino-1-(β-hydroxyethyloxy)benzene, 2-amino-4-(β-hydroxyethylamino)-1-methoxybenzene, 1,3-diaminobenzene, 1,3-bis(2,4-diaminophenoxy)propane, 3-ureidoaniline, 3-ureido-1-dimethylaminobenzene, sesamol, 1-β-hydroxyethylamino-3,4-methylenedioxybenzene, α-naphthol, 2-methyl-1-naphthol, 6-hydroxyindole, 4-hydroxyindole, 4-hydroxy-N-methylindole, 5-methoxy-6-hydroxyindole, 2-amino-3-hydroxypyridine, 6-hydroxybenzomorpholine, 2-amino-4-hydroxyethylaminoanisole, 3-amino-6-methoxy-2-methylaminopyridine, 3,5-diamino-2,6-dimethoxypyridine, 1-N-(β-hydroxyethyl)amino-3,4-methylenedioxybenzene, 2,6-bis(β-hydroxyethylamino)toluene, 6-hydroxyindoline, 2,6-dihydroxy-4-methylpyridine, 2-chloro-3,5-diaminopyridine, 2-chloro-3,5-diamino-6-methoxypyridine, 2-chloro-3,5-diamino-6-methylpyridine, 1-H-3-methylpyrazol-5-one, 1-phenyl-3-methylpyrazol-5-one, 4-(3,5-diaminopyridin-2-yl)-1-(2-hydroxyethyl)-1-methylpiperazin-1-ium chloride, 2,6-dimethylpyrazolo[1,5-b]-1,2,4-triazole, 2,4,6-trimethoxyaniline hydrochloride, 2,6-dimethyl[3,2-c]-1,2,4-triazole, 6-methylpyrazolo[1,5-a]benzimidazole and 2,6-diaminopyrazine, the addition salts thereof and/or the solvates thereof, and mixtures thereof.

Preferably, the coupler(s) used in the process of the invention are chosen from 1,3-dihydroxybenzene, 1,3-dihydroxy-2-methylbenzene, 4-chloro-1,3-dihydroxybenzene, 1-hydroxy-3-aminobenzene, 1-methyl-2-hydroxy-4-β-hydroxyethylaminobenzene, 4-amino-2-hydroxytoluene, 5-amino-6-chloro-2-methylphenol, 2,4-diamino-1-(β-hydroxyethyloxy)benzene, α-naphthol, 6-hydroxyindole, 2-amino-3-hydroxypyridine, 6-hydroxybenzomorpholine, 3-amino-6-methoxy-2-methylaminopyridine and 2-amino-4-hydroxyethylaminoanisole, the addition salts thereof and/or the solvates thereof, and mixtures thereof.

Even more preferably, the coupler(s) used in the process of the invention are chosen from 3-amino-6-methoxy-2-methylaminopyridine, 6-hydroxybenzomorpholine, 2,4-diamino-1-(β-hydroxyethyloxy)benzene, 2-amino-3-hydroxypyridine, 5-amino-6-chloro-2-methylphenol, 1-methyl-2-hydroxy-4-β-hydroxyethylaminobenzene and 2-amino-4-hydroxyethylaminoanisole, the addition salts thereof and/or the solvates thereof, and mixtures thereof.

In general, the addition salts of the couplers that may be used within the context of the invention are notably chosen from the addition salts with an acid such as the hydrochlorides, hydrobromides, sulfates, citrates, succinates, tartrates, lactates, tosylates, benzenesulfonates, phosphates and acetates.

The oxidation bases may be chosen from para-phenylenediamines, bis(phenyl)alkylenediamines, para-aminophenols, ortho-aminophenols and heterocyclic bases, and the addition salts thereof.

Among the para-phenylenediamines, examples that may be mentioned include para-phenylenediamine, para-toluylenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxyethyl)-para-phenylenediamine, 4-N,N-bis(β-hydroxyethyl)amino-2-methylaniline, 4-N,N-bis(β-hydroxyethyl)amino-2-chloroaniline, 2β-hydroxyethyl-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylenediamine, N,N-dimethyl-3-methyl-para-phenylenediamine, N,N-(ethyl-p-hydroxyethyl)-para-phenylenediamine, N-(β,γ-dihydroxypropyl)-para-phenylenediamine, N-(4′-aminophenyl)-para-phenylenediamine, N-phenyl-para-phenylenediamine, 2-β-hydroxyethyloxy-para-phenylenediamine, 2-β-acetylaminoethyloxy-para-phenylenediamine, N-(β-methoxyethyl)-para-phenylenediamine, 4-aminophenylpyrrolidine, 2-thienyl-para-phenylenediamine, 2-β-hydroxyethylamino-5-aminotoluene and 3-hydroxy-1-(4′-aminophenyl)pyrrolidine, and the addition salts thereof with an acid.

Among the para-phenylenediamines mentioned above, para-phenylenediamine, para-toluylenediamine, 2-isopropyl-para-phenylenediamine, 2-β-hydroxyethyl-para-phenylenediamine, 2-β-hydroxyethyloxy-para-phenylenediamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine, 2-chloro-para-phenylenediamine and 2-β-acetylaminoethyloxy-para-phenylenediamine, and the addition salts thereof with an acid, are particularly preferred.

Among the bis(phenyl)alkylenediamines, examples that may be mentioned include N,N′-bis(β-hydroxyethyl)-N,N′-bis(4′-aminophenyl)-1,3-diaminopropanol, N,N′-bis(o-hydroxyethyl)-N,N′-bis(4′-aminophenyl)ethylenediamine, N,N′-bis(4-aminophenyl)tetramethylenediamine, N,N′-bis(β-hydroxyethyl)-N,N′-bis(4-aminophenyl)tetramethylenediamine, N,N′-bis(4-methylaminophenyl)tetramethylenediamine, N,N′-bis(ethyl)-N,N′-bis(4′-amino-3′-methylphenyl)ethylenediamine and 1,8-bis(2,5-diaminophenoxy)-3,6-dioxaoctane, and the addition salts thereof.

Among the para-aminophenols, examples that may be mentioned include para-aminophenol, 4-amino-3-methylphenol, 4-amino-3-fluorophenol, 4-amino-3-chlorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)phenol and 4-amino-2-fluorophenol, and the addition salts thereof with an acid.

Among the ortho-aminophenols, examples that may be mentioned include 2-aminophenol, 2-amino-5-methylphenol, 2-amino-6-methylphenol, 5-acetamido-2-aminophenol and the addition salts thereof.

Among the heterocyclic bases, examples that may be mentioned include pyridine derivatives, pyrimidine derivatives and pyrazole derivatives.

Among the pyridine derivatives, mention may be made of the compounds described for example in patents GB 1 026 978 and GB 1 153 196, for instance 2,5-diaminopyridine, 2-(4-methoxyphenyl)amino-3-aminopyridine and 3,4-diaminopyridine, and addition salts thereof Other pyridine oxidation bases that are useful in the present invention are the 3-aminopyrazolo[1,5-a]pyridine oxidation bases or the addition salts thereof described, for example, in patent application FR 2 801 308. Mention may be made, by way of example, of pyrazolo[1,5-a]pyrid-3-ylamine, 2-(acetylamino)pyrazolo[1,5-a]pyrid-3-ylamine, 2-(morpholin-4-yl)pyrazolo[1,5-a]pyrid-3-ylamine, 3-aminopyrazolo[1,5-a]pyridine-2-carboxylic acid, 2-methoxypyrazolo[1,5-a]pyrid-3-ylamine, (3-aminopyrazolo[1,5-a]pyrid-7-yl)methanol, 2-(3-aminopyrazolo[1,5-a]pyrid-5-yl)ethanol, 2-(3-aminopyrazolo[1,5-a]pyrid-7-yl)ethanol, (3-aminopyrazolo[1,5-a]pyrid-2-yl)methanol, 3,6-diaminopyrazolo[1,5-a]pyridine, 3,4-diaminopyrazolo[1,5-a]pyridine, pyrazolo[1,5-a]pyridine-3,7-diamine, 7-(morpholin-4-yl)pyrazolo[1,5-a]pyrid-3-ylamine, pyrazolo[1,5-a]pyridine-3,5-diamine, 5-(morpholin-4-yl)pyrazolo[1,5-a]pyrid-3-ylamine, 2-[(3-aminopyrazolo[1,5-a]pyrid-5-yl)(2-hydroxyethyl)amino]ethanol, 2-[(3-aminopyrazolo[1,5-a]pyrid-7-yl)(2-hydroxyethyl)amino]ethanol, 3-aminopyrazolo[1,5-a]pyridin-5-ol, 3-aminopyrazolo[1,5-a]pyridin-4-ol, 3-aminopyrazolo[1,5-a]pyridin-6-ol and 3-aminopyrazolo[1,5-a]pyridin-7-ol, and the addition salts thereof.

Among the pyrimidine derivatives, mention may be made of the compounds described, for example, in patents DE 2359399, JP 88-169571, JP 05-63124 and EP 0 770 375 or patent application WO 96/15765, such as 2,4,5,6-tetraaminopyrimidine, 4-hydroxy-2,5,6-triamino-pyrimidine, 2-hydroxy-4,5,6-triaminopyrimidine, 2,4-dihydroxy-5,6-diaminopyrimidine, 2,5,6-triaminopyrimidine and addition salts thereof, and the tautomeric forms thereof, when a tautomeric equilibrium exists.

Among the pyrazole derivatives that may be mentioned are the compounds described in patents DE 3843892 and DE 4133957 and patent applications WO 94/08969, WO 94/08970, FR-A-2 733 749 and DE 195 43 988, such as 4,5-diamino-1-methylpyrazole, 4,5-diamino-1-(β-hydroxyethyl)pyrazole, 3,4-diaminopyrazole, 4,5-diamino-1-(4′-chlorobenzyl)pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl-3-methylpyrazole, 4,5-diamino-1-(β-hydroxyethyl)-3-methylpyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2′-aminoethyl)amino-1,3-dimethylpyrazole, 3,4,5-triaminopyrazole, 1-methyl-3,4,5-triaminopyrazole, 3,5-diamino-1-methyl-4-methylaminopyrazole and 3,5-diamino-4-(β-hydroxyethyl)amino-1-methylpyrazole, and the addition salts thereof. Use may also be made of 4,5-diamino-1-(β-methoxyethyl)pyrazole.

Use will preferably be made of a 4,5-diaminopyrazole and even more preferably of 4,5-diamino-1-(β-hydroxyethyl)pyrazole and/or a salt thereof.

Pyrazole derivatives that may also be mentioned include diamino-N,N-dihydropyrazolopyrazolones and notably those described in patent application FR-A-2 886 136, such as the following compounds and the addition salts thereof: 2,3-diamino-6,7-dihydro-1H,5H-pyrazolo[1,2-a]pyrazol-1-one, 2-amino-3-ethylamino-6,7-dihydro-1H,5H-pyrazolo[1,2-a]pyrazol-1-one, 2-amino-3-isopropylamino-6,7-dihydro-1H,5H-pyrazolo[1,2-a]pyrazol-1-one, 2-amino-3-(pyrrolidin-1-yl)-6,7-dihydro-1H,5H-pyrazolo[1,2-a]pyrazol-1-one, 4,5-diamino-1,2-dimethyl-1,2-dihydropyrazol-3-one, 4,5-diamino-1,2-diethyl-1,2-dihydropyrazol-3-one, 4,5-diamino-1,2-bis(2-hydroxyethyl)-1,2-dihydropyrazol-3-one, 2-amino-3-(2-hydroxyethyl)amino-6,7-dihydro-1H,5H-pyrazolo[1,2-a]pyrazol-1-one, 2-amino-3-dimethylamino-6,7-dihydro-1H,5H-pyrazolo[1,2-a]pyrazol-1-one, 2,3-diamino-5,6,7,8-tetrahydro-1H,6H-pyridazino[1,2-a]pyrazol-1-one, 4-amino-1,2-diethyl-5-(pyrrolidin-1-yl)-1,2-dihydropyrazol-3-one, 4-amino-5-(3-dimethylaminopyrrolidin-1-yl)-1,2-diethyl-1,2-dihydropyrazol-3-one, 2,3-diamino-6-hydroxy-6,7-dihydro-1H,5H-pyrazolo[1,2-a]pyrazol-1-one.

Use will preferably be made of 2,3-diamino-6,7-dihydro-1H,5H-pyrazolo[1,2-a]pyrazol-1-one and/or a salt thereof.

Heterocyclic bases that will preferably be used include 4,5-diamino-1-(β-hydroxyethyl)pyrazole and/or 2,3-diamino-6,7-dihydro-1H,5H-pyrazolo[1,2-a]pyrazol-1-one and/or a salt thereof.

Preferably, the oxidation dyes are chosen from heterocyclic bases and heterocyclic or benzene-based couplers with at least one amine substituent on the aromatic ring.

Preferably, the oxidation dyes are chosen from couplers and more preferably heterocyclic or benzene-based couplers with at least one amine substituent on the aromatic ring.

The term “direct dye” means natural and/or synthetic dyes, other than oxidation dyes. They are dyes that will superficially diffuse on the fibre and dye the fibres by themselves.

The direct dye(s) may be natural or synthetic, cationic, anionic or neutral direct dyes.

Mention may be made, as a direct dye according to the invention, of the following dyes: acridines; acridones; anthranthrones; anthrapyrimidines; anthraquinones; azines; (poly)azos, hydrazono or hydrazones, in particular arylhydrazones; azomethines; benzanthrones; benzimidazoles; benzimidazolones; benzindoles; benzoxazoles; benzopyrans; benzothiazoles; benzoquinones; bisazines; bis-isoindolines; carboxanilides; coumarins; cyanines, such as azacarbocyanines, diazacarbocyanines, diazahemicyanines, hemicyanines or tetraazacarbocyanines; diazines; diketopyrrolopyrroles; dioxazines; diphenylamines; diphenylmethanes; dithiazines; flavonoids, such as flavanthrones and flavones; fluorindines; formazans; indamines; indanthrones; indigoids and pseudoindigoids; indophenols; indoanilines; isoindolines; isoindolinones; isoviolanthrones; lactones; (poly)methines, such as dimethines of stilbene or styryl types; naphthalimides; naphthanilides; naphtholactams; naphthoquinones; nitro, in particular nitro(hetero)aromatics; oxadiazoles; oxazines; perilones; perinones; perylenes; phenazines; phenoxazines; phenothiazines; phthalocyanines; polyenes/carotenoids; porphyrins; pyranthrones; pyrazolanthrones; pyrazolones; pyrimidinoanthrones; pyronines; quinacridones; quinolines; quinophthalones; squaranes; tetrazoliums; thiazines; thioindigos; thiopyronines; triarylmethanes or xanthenes.

More particularly, the direct dyes of the invention are chosen from neutral, acidic or cationic nitrobenzene direct dyes, neutral, acidic or cationic azo direct dyes, tetraazapentamethine dyes, neutral, acidic or cationic quinone and in particular anthraquinone dyes, azine direct dyes, triarylmethane direct dyes, azomethine direct dyes and natural direct dyes.

According to one embodiment, the direct dyes of the invention are anionic. The latter are dyes commonly called “acidic dyes” for their affinity with alkaline substances (see, for example, “Industrial Dyes, Chemistry, Properties, Applications”, Klaus Hunger ed., Wiley-VCH Verlag GmbH & Co. KGaA, Weinheim 2003). Anionic or acidic dyes are known in the literature (see, for example, Ullman's Encyclopedia of Industrial Chemistry, Azo Dyes, 2005 Wiley-VCH Verlag GmbH & Co. KGaA, Weinheim 10.1002/14356007.a03 245, point 3.2; ibid, Textile Auxiliaries, 2002 Wiley-VCH Verlag GmbH & Co. KGaA, Weinheim 10.1002/14356007.a26 227 and Ashford's Dictionary of Industrial Chemicals, Second Edition, p. 14-p. 39, 2001).

The term “anionic direct dye” means any direct dye comprising in its structure at least one sulfonate group SO₃— and/or at least one carboxylate group C(O)O— and optionally one or more anionic groups G- with G-, which may be identical or different, representing an anionic group chosen from alkoxide O—, thiolate

S—, carboxylate and thiocarboxylate: C(Q)Q′-, with Q and Q′, which may be identical or different, representing an oxygen or sulfur atom; preferably, G- represents a carboxylate, i.e. Q and Q′ represents an oxygen atom.

The preferred anionic dyes are chosen from acidic nitro direct dyes, acidic azo dyes, acidic azine dyes, acidic triarylmethane dyes, acidic indoamine dyes, acidic anthraquinone dyes, indigoids and acidic natural dyes, each of these dyes containing at least one sulfonate or carboxylate group.

As anionic dyes according to the invention, mention may be made of the dyes of formulae (II), (II′), (III), (III′), (IV), (IV′), (V), (V′), (VI), (VII), (VIII) and (IX) below:

a) the diaryl anionic azo dyes of formula (II) or (II′):

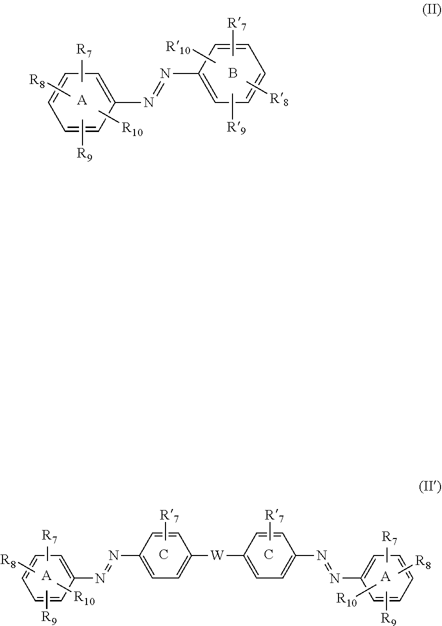

in which formulae (II) and (II′):
R7, R8, R9, R10, R′7, R′8, R′9 and R′10, which may be identical or different, represent a hydrogen atom or a group chosen from:
alkyl;
alkoxy, alkylthio;
hydroxyl, mercapto;
nitro;
R^{o}—C(X)—X′—, R^{o}—X′—C(X)—, R^{o}—X′—C(X)—X″— with R^{o} representing a hydrogen atom or an alkyl or aryl group; X, X′ and X″, which may be identical or different, representing an oxygen or sulfur atom, or NR with R representing a hydrogen atom or an alkyl group;
(O)₂S(O⁻)—, M⁺ with M⁺ representing a cationic counterion such as an alkali metal (Na, K) or an alkaline-earth metal (Ca);
(O)CO⁻—, M⁺ with M⁺ as defined previously;
R″—S(O)₂—, with R″ representing a hydrogen atom or an alkyl, aryl, (di)(alkyl)amino or aryl(alkyl)amino group; preferably a phenylamino or phenyl group;
R′″—S(O)₂—X′— with R′″ representing an alkyl or optionally substituted aryl group, X′ as defined previously;
(di)(alkyl)amino;
aryl(alkyl)amino optionally substituted with one or more groups chosen from i) nitro; ii) nitroso; iii) (O)₂S(O⁻)—, M⁺ and iv) alkoxy with M⁺ as defined previously;
optionally substituted heteroaryl; preferably a benzothiazolyl group;
cycloalkyl; especially cyclohexyl;
Ar—N═N— with Ar representing an optionally substituted aryl group, preferably a phenyl optionally substituted with one or more alkyl, (O)₂S(O⁻)—, M⁺ or phenylamino groups;
or alternatively two contiguous groups R7 with R8 or R8 with R9 or R9 with R10 together form a fused benzo group A′; and R′7 with R′8 or R′8 with R′9 or R′9 with R′10 together form a fused benzo group B′; with A′ and B′ optionally substituted with one or more groups chosen from i) nitro; ii) nitroso; iii) (O)₂S(O⁻)—, M₊; iv) hydroxyl; v) mercapto; vi) (di)(alkyl)amino; vii) R^{o}—C(X)—X′—; viii) R^{o}—X′—C(X)—; ix) R^{o}—X′—C(X)—X″—; x) Ar—N═N— and xi) optionally substituted aryl(alkyl)amino; with M⁺, R^{o}, X, X′, X″ and Ar as defined previously;
W represents a sigma bond a, an oxygen or sulfur atom, or a divalent radical i) —NR— with R as defined previously, or ii) methylene —C(Ra)(Rb)- with Ra and Rb, which may be identical or different, representing a hydrogen atom or an aryl group, or alternatively Ra and Rb together form, with the carbon atom that bears them, a spiro cycloalkyl; preferably W represents a sulfur atom or Ra and Rb together form a cyclohexyl;

it being understood that formulae (II) and (II′) comprise at least one sulfonate (O)₂S(O⁻) —, M⁺ or carboxylate (O)C(O—)—, M⁺ radical on one of the rings A, A′, B, B′ or C with M⁺ as defined previously;

By way of example of dyes of formula (II), mention may be made of Acid Red 1, Acid Red 4, Acid Red 13, Acid Red 14, Acid Red 18, Acid Red 27, Acid Red 32, Acid Red 33, Acid Red 35, Acid Red 37, Acid Red 40, Acid Red 41, Acid Red 42, Acid Red 44, Acid Red 68, Acid Red 73, Acid Red 135, Acid Red 138, Acid Red 184, Food Red 1, Food Red 13, Acid Orange 6, Acid Orange 7, Acid Orange 10, Acid Orange 19, Acid Orange 20, Acid Orange 24, Acid Yellow 9, Acid Yellow 36, Acid Yellow 199, Food Yellow 3; Acid Violet 7, Acid Violet 14, Acid Blue 113, Acid Blue 117, Acid Black 1, Acid Brown 4, Acid Brown 20, Acid Black 26, Acid Black 52, Food Black 1 and Food Black 2;

and as examples of dyes of formula (II′), mention may be made of Acid Red 111, Acid Red 134 and Acid Yellow 38;

b) the pyrazolone anionic azo dyes of formulae (III) and (III′):

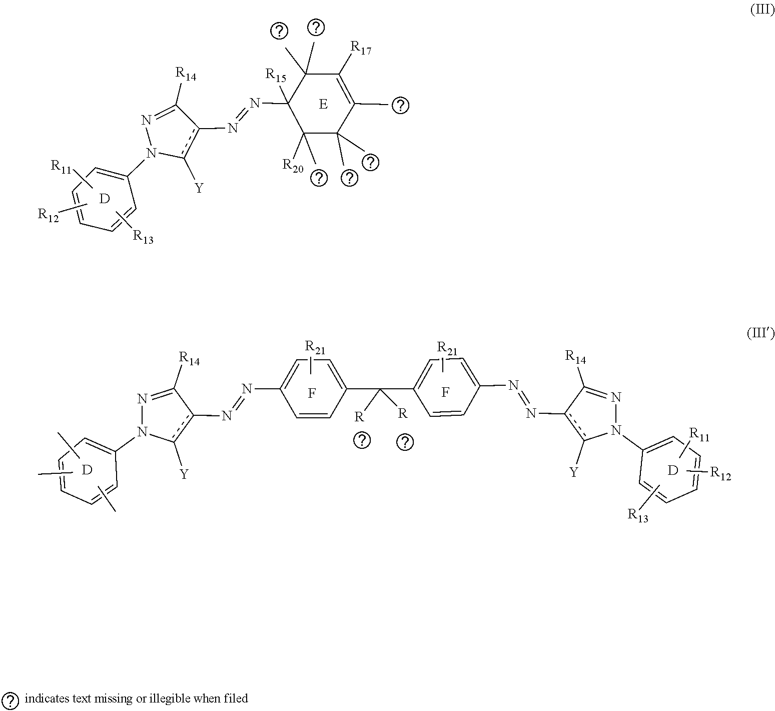

in which formulae (III) and (III′):
R11, R12 and R13, which may be identical or different, represent a hydrogen or halogen atom, an alkyl group or —(O)₂S(O⁻), M⁺ with M⁺ as defined previously;
R14 represents a hydrogen atom, an alkyl group or a group —C(O)O—, M⁺ with M⁺ as defined previously;
R15 represents a hydrogen atom;
R16 represents an oxo group, in which case R′16 is absent, or alternatively R15 with R16 together form a double bond;
R17 and R18, which may be identical or different, represent a hydrogen atom, or a group chosen from:
(O)₂S(O⁻)—, M⁺ with M⁺ as defined above;
Ar—O—S(O)₂— with Ar representing an optionally substituted aryl group; preferably a phenyl optionally substituted with one or more alkyl groups;
R19 and R20 together form either a double bond, or a benzo group D′, which is optionally substituted;
R′16, R′19 and R′20, which may be identical or different, represent a hydrogen atom or an alkyl or hydroxyl group;
R21 represents a hydrogen atom or an alkyl or alkoxy group;
Ra and Rb, which may be identical or different, are as defined previously, preferably Ra represents a hydrogen atom and Rb represents an aryl group;
Y represents either a hydroxyl group or an oxo group;

[symbol] represents a single bond when Y is an oxo group; and represents a double bond when Y represents a hydroxyl group;

it being understood that formulae (III) and (III′) comprise at least one sulfonate group (O)₂S(O⁻)—, M⁺ on one of the rings D or E or formulae (III) and (III′) comprise at least one carboxylate group (O)C(O⁻)—, M⁺ with M⁺ as defined previously;

As examples of dyes of formula (III), mention may be made of Acid Red 195, Acid Yellow 23, Acid Yellow 27, Acid Yellow 76, and, as examples of dyes of formula (III′), mention may be made of the ammonium salt derived from Acid Yellow 17;

c) the anthraquinone dyes of formulae (IV) and (IV′):

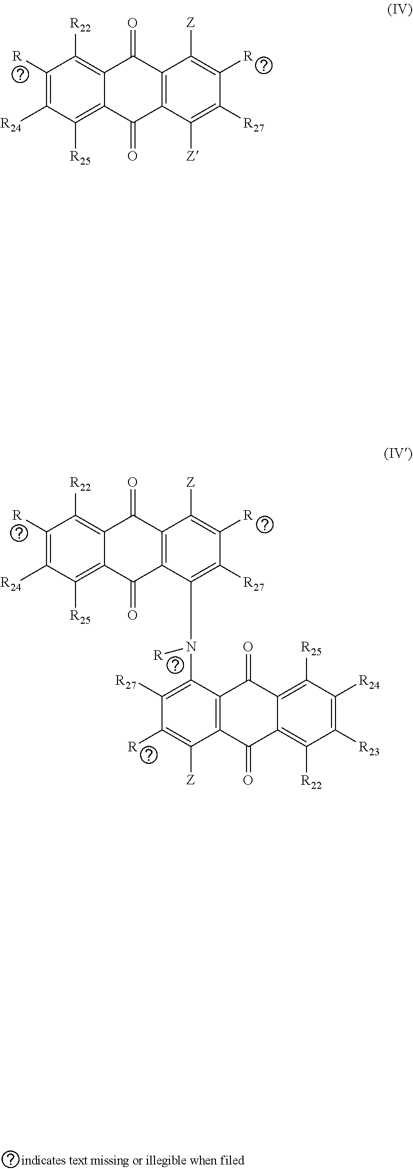

in which formulae (IV) and (IV′):
R22, R23, R24, R25, R26 and R27, which may be identical or different, represent a hydrogen or halogen atom, or a group chosen from:
alkyl;
hydroxyl, mercapto;
alkoxy, alkylthio;
optionally substituted aryloxy or arylthio, preferably substituted with one or more groups chosen from alkyl and (O)₂S(O⁻)—, M⁺ with M⁺ as defined previously;
aryl(alkyl)amino optionally substituted with one or more groups chosen from alkyl and (O)₂S(O⁻)—, M⁺ with M⁺ as defined previously;
(di)(alkyl)amino;
(di)(hydroxyalkyl)amino;
(O)₂S(O⁻)—, M⁺ with M⁺ as defined above;
Z′ represents a hydrogen atom or a group NR28R29 with R28 and R29, which may be identical or different, representing a hydrogen atom or a group chosen from:

alkyl;
polyhydroxyalkyl such as hydroxyethyl;
aryl optionally substituted with one or more groups, particularly i) alkyl, such as methyl, n-dodecyl, n-butyl; ii) (O)₂S(O⁻)—, M⁺ with M⁺ as defined previously; iii) R^{o}—C(X)—X′—, R^{o}—X′—C(X)—, R^{o}—X′—C(X)—X″— with R^{o}, X, X′ and X″ as defined previously, preferentially R^{o} represents an

alkyl group;
cycloalkyl; in particular cyclohexyl;
Z represents a group chosen from hydroxyl and NR′28R′29 with R′28 and R′29, which may be identical or different, representing the same atoms or groups as R28 and R29 as defined previously;
it being understood that formulae (IV) and (IV′) comprise at least one sulfonate group (O)₂S(O⁻)—, M⁺ with M⁺ as defined previously.

As examples of dyes of formula (IV), mention may be made of Acid Blue 25, Acid Blue 43, Acid Blue 62, Acid Blue 78, Acid Blue 129, Acid Blue 138, Acid Blue 140, Acid Blue 251, Acid Green 25, Acid Green 41, Acid Violet 42, Acid Violet 43 and Mordant Red 3;

and as examples of dyes of formula (IV′), mention may be made of the ammonium salt derived from Acid Black 48;

d) the dyes corresponding to formulae (V) and (V′) below:

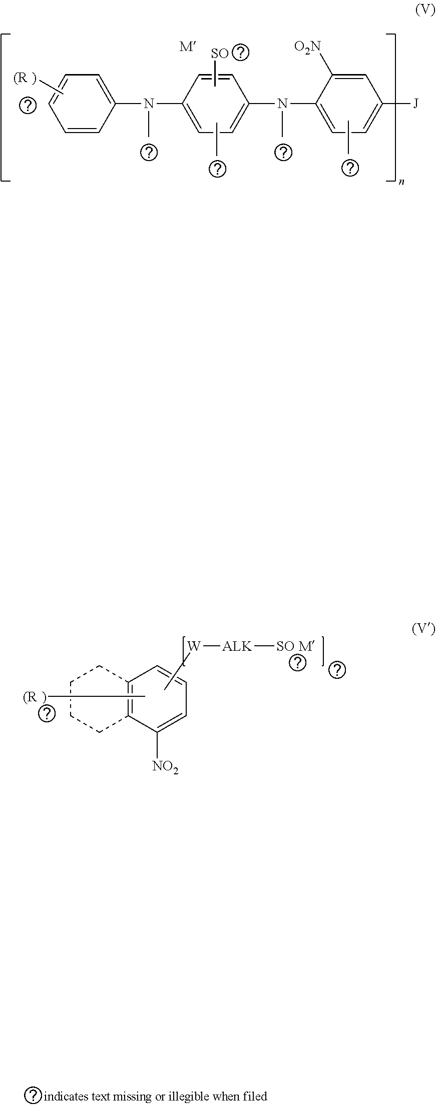

in which formulae (V) and (V′):
R30, R31 and R32, which may be identical or different, represent a hydrogen or halogen atom, or a group chosen from:
alkyl;
alkoxy optionally substituted with one or more hydroxyl groups, alkylthio optionally substituted with one or more hydroxyl groups;
hydroxyl, mercapto;
nitro, nitroso;
(poly)haloalkyl;
R^{o}—C(X)—X′—, R^{o}—X′—C(X)—, R^{o}—X′—C(X)—X″— with R^{o}, X, X′ and X″ as defined above;
(O)₂S(O⁻)—, M⁺ with M⁺ as defined above;
(O)CO⁻—, M⁺ with M⁺ as defined above;
(di)(alkyl)amino;
(di)(hydroxyalkyl)amino;
heterocycloalkyl such as piperidino, piperazino or morpholino;

in particular, R30, R31 and R32 represent a hydrogen atom;
Rc and Rd, which may be identical or different, represent a hydrogen atom or an alkyl group;
W is as defined previously; W particularly represents an —NH— group;
ALK represents a linear or branched divalent C₁-C₆ alkylene group; in particular, ALK represents a group —CH₂—CH₂—;
n is 1 or 2;
p represents an integer inclusively between 1 and 5;
q represents an integer inclusively between 1 and 4;
u is 0 or 1;
when n is 1, J represents a nitro or nitroso group; particularly nitro;
when n is 2, J represents an oxygen or sulfur atom, or a divalent radical —S(O)ₘ— with m representing an integer 1 or 2; preferably, J represents an —SO₂— radical;
M′ is as defined above for M⁺;

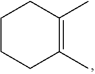

which may be present or absent, represents a benzo group optionally substituted with one or more groups R30 as defined above;

it being understood that formulae (V) and (V′) comprise at least one sulfonate group (O)₂S(O⁻)—, M⁺ or carboxylate group (O)C(O⁻)—, M⁺ with M⁺ as defined previously;

As examples of dyes of formula (V), mention may be made of: Acid Brown 13 and Acid Orange 3; as examples of dyes of formula (V′), mention may be made of: Acid Yellow 1, sodium salt of 2,4-dinitro-1-naphthol-7-sulfonic acid, 2-piperidino-5-nitrobenzenesulfonic acid, 2-(4′-N,N(2″-hydroxyethyl)amino-2′-nitro)anilineethanesulfonic acid and 4-β-hydroxyethylamino-3-nitrobenzenesulfonic acid;

e) the triarylmethane dyes of formula (VI):

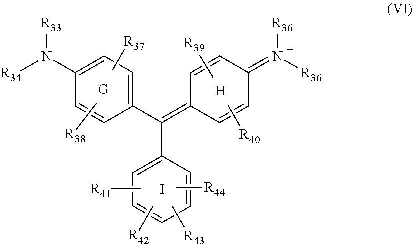

in which formula (VI):
R33, R34, R35 and R36, which may be identical or different, represent a hydrogen atom or a group chosen from alkyl, optionally substituted aryl and optionally substituted arylalkyl; particularly an alkyl group and benzyl optionally substituted with a group (O)ₘS(O⁻)—, M⁺ with M⁺ and m as defined previously;
R37, R38, R39, R40, R41, R42, R43 and R44, which may be identical or different, represent a hydrogen atom or a group chosen from:
alkyl;
alkoxy, alkylthio;
(di)(alkyl)amino;
hydroxyl, mercapto;
nitro, nitroso;
R^{o}—C(X)—X′—, R^{o}—X′—C(X)—, R^{o}—X′—C(X)—X″— with R^{o} representing a hydrogen atom or an alkyl or aryl group; X, X′ and X″, which may be identical or different, representing an oxygen or sulfur atom, or NR with R representing a hydrogen atom or an alkyl group;
(O)₂S(O⁻)—, M⁺ with M⁺ representing a hydrogen atom or a cationic counterion;
(O)CO⁻—, M⁺ with M⁺ as defined above;
or alternatively two contiguous groups R41 with R42 or R42 with R43 or R43 with R44 together form a fused benzo group: I′; with I′ optionally substituted with one or more groups chosen from i) nitro; ii) nitroso; iii) (O)₂S(O⁻)—, M⁺; iv) hydroxyl; v) mercapto; vi) (di)(alkyl)amino; vii) R^{o}—C(X)—X′—; viii) R^{o}—X′—C(X)—; ix) R^{o}—X′—C(X)—X″—; with M⁺, R^{o}, X, X′, X″ as defined above;

particularly, R37 to R40 represent a hydrogen atom, and R41 to R44, which may be identical or different, represent a hydroxyl group or (O)₂S(O⁻)—, M⁺; and when R43 with R44 together form a benzo group, it is preferably substituted with a group (O)₂S(O⁻)—;

it being understood that at least one of the rings G, H, I or I′ comprises at least one sulfonate (O)₂S(O⁻)—, M⁺ or carboxylate (O)C(O⁻)—, M⁺ group as defined previously;

As examples of dyes of formula (VI), mention may be made of: Acid Blue 1; Acid Blue 3; Acid Blue 7, Acid Blue 9; Acid Violet 49 and Acid Green 50;

f) the xanthene-based dyes of formula (VII):

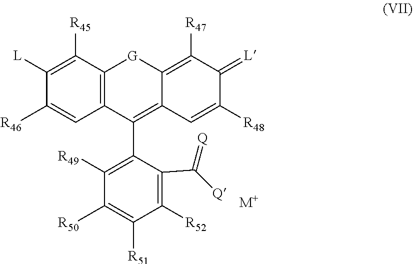

in which formula (VII):
R45, R46, R47 and R48, which may be identical or different, represent a hydrogen atom or a halogen atom;
R49, R50, R51 and R52, which may be identical or different, represent a hydrogen atom, a halogen atom or a group chosen from:
alkyl;
alkoxy, alkylthio;
hydroxyl, mercapto;
nitro, nitroso;
(O)₂S(O⁻)—, M⁺ with M⁺ representing a hydrogen atom or a cationic counterion;
(O)CO⁻—, M⁺ with M⁺ as defined above;
particularly R53, R54, R55 and R48 represent a hydrogen or halogen atom;
G represents an oxygen or sulfur atom or a group NRe with Re as defined previously; particularly, G represents an oxygen atom;
L represents an alkoxide O⁻, M⁺; a thioalkoxide S⁻, M⁺ or a group NRf, with Rf representing a hydrogen atom or an alkyl group, and M⁺ as defined previously; M⁺ is particularly sodium;
L′ represents an oxygen or sulfur atom or an ammonium group: N+RfRg, with Rf and Rg, which may be identical or different, representing a hydrogen atom, an alkyl group or an aryl group, optionally substituted; L′ represents particularly an oxygen atom or a phenylamino group optionally substituted with one or more alkyl or (O)ₘS(O⁻)—, M⁺ groups with m and M⁺ as defined previously;
Q and Q′, which may be identical or different, represent an oxygen or sulfur atom; particularly Q and Q′ represent an oxygen atom;
M⁺ is as defined above;

it being understood that formula (VII) comprises at least one sulfonate group (O)₂S(O⁻)—, M⁺ or carboxylate group (O)C(O⁻)—, M⁺ with M⁺ as defined previously;

As examples of dyes of formula (VII), mention may be made of: Acid Yellow 73; Acid Red 51; Acid Red 87; Acid Red 92; Acid Red 95 and Acid Violet 9;

g) the indole-based dyes of formula (VIII):

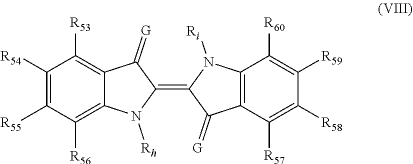

R53, R54, R55, R56, R57, R58, R59 and R60, which may be identical or different, represent a hydrogen atom or a group chosen from:
alkyl;
alkoxy, alkylthio;
hydroxyl, mercapto;
nitro, nitroso;
R^{o}—C(X)—X′—, R^{o}—X′—C(X)—, R^{o}—X′—C(X)—X″— with R^{o} representing a hydrogen atom or an alkyl or aryl group; X, X′ and X″, which may be identical or different, representing an oxygen or sulfur atom, or NR with R representing a hydrogen atom or an alkyl group;
(O)₂S(O⁻)—, M⁺ with M⁺ as defined above;
(O)CO⁻—, M⁺ with M⁺ as defined above;
G represents an oxygen or sulfur atom or a group NRe with Re as defined previously; particularly G represents an oxygen atom;
Ri and Rh, which may be identical or different, represent a hydrogen atom or an alkyl group;

it being understood that formula (VIII) comprises at least one sulfonate group (O)₂S(O⁻)—, M⁺ or carboxylate group (O)C(O)—, M⁺ with Was defined above;

As examples of dyes of formula (VIII), mention may be made of the ammonium salt derived from: Acid Blue 74;

h) the quinoline-based dyes of formula (IX):

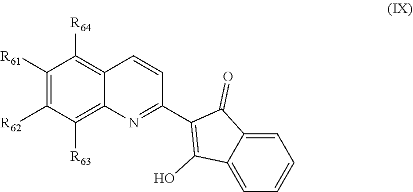

R61 represents a hydrogen or halogen atom or an alkyl group;
R62, R63 and R64, which may be identical or different, represent a hydrogen atom or a group (O)₂S(O⁻)—, M⁺ with M⁺ as defined previously;
or alternatively R61 with R62, or R61 with R64, together form a benzo group optionally substituted with one or more groups (O)₂S(O⁻)—, M⁺ with M⁺ representing a hydrogen atom or a cationic counterion;

it being understood that formula (IX) comprises at least one sulfonate group (O)₂S(O⁻)—, with M⁺ as defined previously;

As examples of dyes of formula (IX), mention may be made of: Acid Yellow 2, Acid Yellow 3 and Acid Yellow 5.

More particularly, the anionic direct dyes that are useful in the invention may be chosen from the following dyes:

TABLE 1

| | |
|---|---|
| (C.I. 45380) | Acid Red 87 belonging to formula (VII) |
| (C.I. 10316) | salts of 2,4-dinitro-1-naphthol-7-sulfonic acid belonging to |
| | formula (V′) |
| (C.I. 10383) | Acid Orange 3 belonging to formula (V) |
| (C.I. 13015) | Acid Yellow 9/Food Yellow 2 belonging to formula (II) |
| (C.I. 14780) | Direct Red 45/Food Red 13 belonging to formula (II) |
| (C.I. 13711) | Acid Black 52 belonging to formula (II) |
| (C.I. 13065) | Acid Yellow 36 belonging to formula (II) |
| (C.I. 14700) | salt of 1-hydroxy-2-(2′,4′-xylyl-5-sulfonatoazo)naphthalene- |
| | 4-sulfonic acid/Food Red 1 belonging to formula (II) |
| (C.I. 14720) | Acid Red 14/Food Red 3/Mordant Blue 79 belonging to |
| | formula (II) |
| (C.I. 14805) | salt of 4-hydroxy-3-[(2-methoxy-5-nitrophenyl)diaza]-6- |
| | (phenylamino)naphthalene-2-sulfonic acid/Acid Brown 4 |
| | belonging to formula (II) |
| (C.I. 15510) | Acid Orange 7/Pigment Orange 17/Solvent Orange 49 |
| | belonging to formula (II) |
| (C.I. 15985) | Food Yellow 3/Pigment Yellow 104 belonging to formula |
| | (II) |
| (C.I. 16185) | Acid Red 27/Food Red 9 belonging to formula (II) |
| (C.I. 16230) | Acid Orange 10/Food Orange 4 belonging to formula (II) |
| (C.I. 16250) | Acid Red 44 belonging to formula (II) |
| (C.I. 17200) | Acid Red 33/Food Red 12 belonging to formula (II) |
| (C.I. 15685) | Acid Red 184 belonging to formula (II) |
| (C.I. 19125) | Acid Violet 3 belonging to formula (II) |
| (C.I. 18055) | salt of 1-hydroxy-2-(4′-acetamidophenylazo)-8- |
| | acetamidonaphthalene-3,6-disulfonic acid/Acid Violet 7/ |
| | Food Red 11 belonging to formula (II) |
| (C.I. 18130) | Acid Red 135 belonging to formula (II) |
| (C.I. 19130) | Acid Yellow 27 belonging to formula (III) |
| (C.I. 19140) | Acid Yellow 23/Food Yellow 4 belonging to formula (III) |
| (C.I. 20170) | 4′-(sulfonato-2″,4″-dimethyl)bis(2,6-phenylazo)-1,3- |
| | dihydroxybenzene/Acid Orange 24 belonging to formula |
| | (II) |
| (C.I. 20470) | salt of 1-amino-2-(4′-nitrophenylazo)-7-phenylazo-8- |
| | hydroxynaphthalene-3,6-disulfonic acid/Acid Black 1 (II) |
| (C.I. 23266) | (4-((4-methylphenyl)sulfonyloxy)phenylazo)-2,2′-dimethyl- |
| | 4-((2-hydroxy-5,8-disulfonato)naphthylazo)biphenyl/Acid |
| | Red 111 belonging to formula (II′) |
| (C.I. 27755) | Food Black 2 belonging to formula (II) |
| (C.I. 25440) | 1-(4′-sulfonatophenylazo)-4-((2″-hydroxy-3″-acetylamino- |
| | 6″,8″-disulfonato)naphthylazo)-6-sulfonatonaphthalene |
| | (tetrasodium salt)/Food Black 1 belonging to formula (II) |
| (C.I. 42090) | Acid Blue 9 belonging to formula (VI) |
| (C.I. 60730) | Acid Violet 43 belonging to formula (IV) |
| (C.I. 61570) | Acid Green 25 belonging to formula (IV) |
| (C.I. 62045) | salt of 1-amino-4-cyclohexylamino-9,10-anthraquinone-2- |
| | sulfonic acid/Acid Blue 62 belonging to formula (IV) |
| (C.I. 62105) | Acid Blue 78 belonging to formula (IV) |
| (C.I. 14710) | salt of 4-hydroxy-3-((2-methoxyphenyl)azo)-1- |
| | naphthalenesulfonic acid/Acid Red 4 belonging to formula |
| | (II) |
| | 2-piperidino-5-nitrobenzenesulfonic acid belonging to |
| | formula (V′) |
| | 2-(4′-N,N-(2″-hydroxyethyl)amino-2′-nitro)anilineethane- |
| | sulfonic acid belonging to formula (V′) |
| | 4-β-hydroxyethylamino-3-nitrobenzenesulfonic acid |
| | belonging to formula (V′) |
| (C.I. 42640) | Acid Violet 49 belonging to formula (VI) |
| (C.I. 42080) | Acid Blue 7 belonging to formula (VI) |
| (C.I. 58005) | ammonium salts of 1,2-dihydroxy-3-sulfoanthraquinone/ |
| | Mordant Red 3 belonging to formula (IV) |
| (C.I. 62055) | salt of 1-amino-9,10-dihydro-9,10-dioxo-4-(phenylamino)- |
| | 2-anthracenesulfonic acid/Acid Blue 25 belonging to |
| | formula (IV) |
| (C.I. 14710) | salt of 4-hydroxy-3-((2-methoxyphenyl)azo)-1- |
| | naphthalenesulfonic acid/Acid Red 4 belonging to formula |
| | (II) |

Most of these dyes are described in particular in the Colour Index published by The Society of Dyers and Colourists, P.O. Box 244, Perkin House, 82 Grattan Road, Bradford, Yorkshire, BD1 2JBN England.

The anionic dyes that are most particularly preferred are the dyes designated in the Colour Index under the code C.I. 58005 (monosodium salt of 1,2-dihydroxy-9,10-anthraquinone-3-sulfonic acid), C.I. 60730 (monosodium salt of 2-[(9,10-dihydro-4-hydroxy-9,10-dioxo-1-anthracenyl)amino]-5-methylbenzenesulfonic acid), C.I. 15510 (monosodium salt of 4-[(2-hydroxy-1-naphthalenyl)azo]benzenesulfonic acid), C.I. 15985 (disodium salt of 6-hydroxy-5-[(4-sulfophenyl)azo]-2-naphthalenesulfonic acid), C.I. 17200 (disodium salt of 5-amino-4-hydroxy-3-(phenylazo)-2,7-naphthalenedisulfonic acid), C.I. 20470 (disodium salt of 1-amino-2-(4′-nitrophenylazo)-7-phenylazo-8-hydroxy-3,6-naphthalenedisulfonic acid), C.I. 42090 (disodium salt of N-ethyl-N-4-[4-[[4-[ethyl[3-sulfophenyl)methyl]amino]phenyl](2-sulfophenyl)methylene]-2,5-cyclohexadien-1-ylidene]-3-sulfobenzenemethanaminium hydroxide, inner salt), C.I. 61570 (disodium salt of 2,2′-[(9,10-dihydro-9,10-dioxo-1,4-anthracenediyl)diimino]bis[5-methyl]benzenesulfonic acid). Use may also be made of compounds corresponding to the mesomeric or tautomeric forms of structures (II) to (IX).

More preferably, the anionic dyes of formula (I) according to the invention are chosen from those of formulae (II), (III) and (IV).

According to a particular embodiment of the invention, the direct dyes of the invention are chosen from those corresponding to formulae (Ha), (IIIa) and (IVa) below:

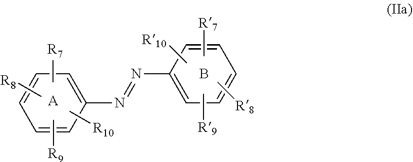

In which formula (Ha):
R7, R8, R9, R10, R′7, R′8, R′9 and R′10, which may be identical or different, represent a hydrogen atom or a group chosen from:
hydroxyl,
nitro, nitroso;
(di)(alkyl)amino;
(O)₂S(O⁻)—, M⁺ with M⁺ representing a hydrogen atom or a cationic counterion as defined previously; and
Ar—N═N— with Ar representing an optionally substituted aryl group; preferably a phenyl optionally substituted with one or more alkyl or (O)₂S(O⁻)—, M⁺ groups;
or alternatively two contiguous groups R7 with R8 or R8 with R9 or R9 with R10 together form a fused benzo group A′; and R′7 with R′8 or R′8 with R′9 or R′9 with R′10 together form a fused benzo group B′; with A′ and B′ optionally substituted with one or more groups chosen from a) (O)₂S(O⁻)—, M⁺; b) hydroxyl; c) Ar—N═N—; with M⁺ and Ar as defined previously;

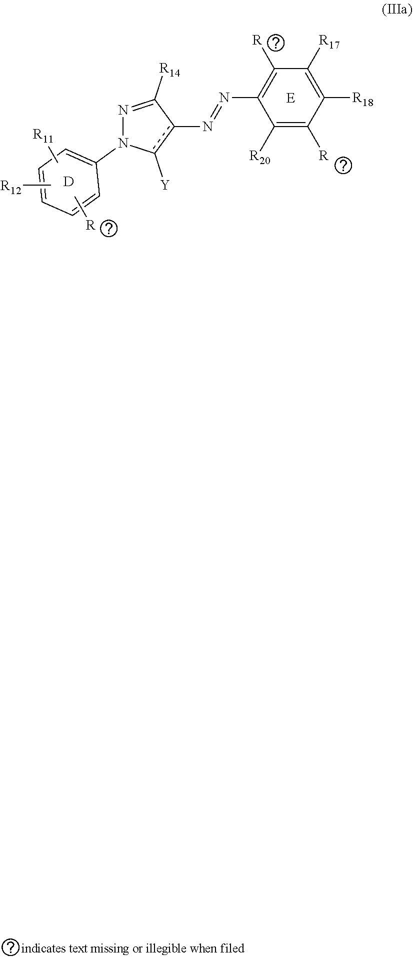

in which formula (Ma):
R11, R12 and R13, which may be identical or different, represent a hydrogen or halogen atom, an alkyl group or —(O)₂S(O⁻), M⁺ with M⁺ as defined previously;
R14 represents a hydrogen atom, an alkyl group or a group —C(O)O—, M⁺ with M⁺ as defined above;
R16, R17, R18, R19 and R20, which may be identical or different, represent a hydrogen atom or an alkyl, hydroxyl or (O)₂S(O⁻)—, M⁺ group with M⁺ as defined previously;
Y represents either a hydroxyl group or an oxo group;
[symbol] represents a single bond when Y is an oxo group; and represents a double bond when Y represents a hydroxyl group;

it being understood that formula (Ma) comprises at least one sulfonate group (O)₂S(O⁻) —, M⁺ on one of the rings D or E or carboxylate (O)C(O⁻)—, M⁺;

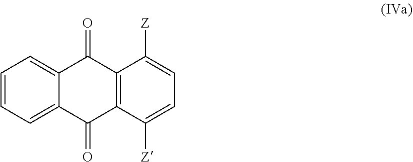

in which formula (IVa):
Z′ represents a group NR28R29 with R28 representing a hydrogen atom or an alkyl group and R29 representing an aryl group optionally substituted particularly with one or more groups chosen from i) alkyl such as methyl and ii) (O)₂S(O⁻)—, M⁺ with M⁺ as defined previously;
Z represents a group chosen from hydroxyl and NR′28R′29 with R′28 and R′29, which may be identical or different, representing the same atoms or groups as R28 and R29 as defined previously;

it being understood that formula (IVa) comprises at least one sulfonate group (O)₂S(O⁻) —, M⁺;

By way of example made of the following anionic dyes:

TABLE 2

| Commercial name | Corresponding structure |
|---|---|
| Acid Orange 7 | 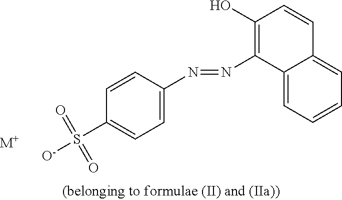 |
| Acid Black 1 | 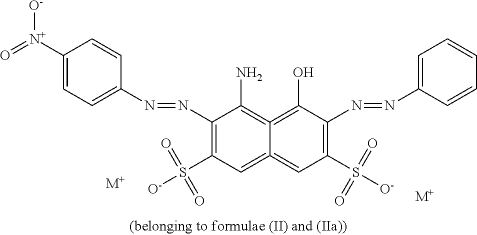 |
| Acid Red 18 | 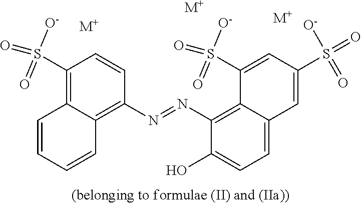 |
| Acid Yellow 23 | 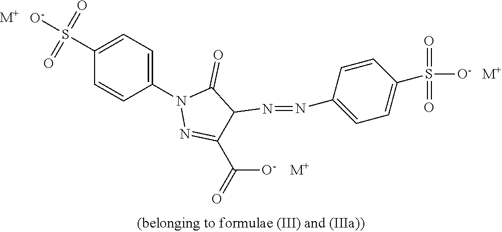 |
| Acid Violet 43 | 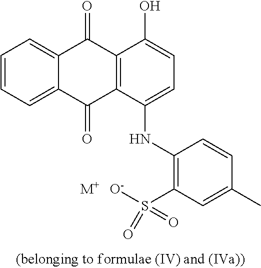 |

With M⁺, which may be identical or different, being a cationic counterion as defined previously.

According to a particular embodiment of the invention, the direct dyes are benzene-based dyes that are usually neutral. More preferably, the benzene-based direct dyes of the invention are chosen from the compounds of formula (X) below:

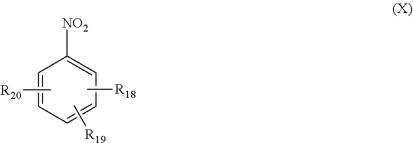

In which formula (X):
R18 represents an amino radical; an amino radical monosubstituted or disubstituted with a C₁-C₄ alkyl radical, C₁-C₄ monohydroxyalkyl radical, C₂-C₄ polyhydroxyalkyl radical, C₁-C₄ aminoalkyl radical, mono(C₁-C₄)alkylamino(C₁-C₄)alkyl radical, di(C₁-C₄)alkylamino(C₁-C₄)alkyl radical, C₁-C₄ ureidoalkyl radical, aryl radical, or aryl radical in which the aryl ring is substituted with one or more hydroxyl, carboxyl, amino or di(C₁-C₄)alkylamino radicals,
R19 represents a hydrogen atom; an amino radical; a hydroxyl radical; a C₁-C₄ alkyl radical; a C₁-C₄ alkoxy radical; a C₁-C₄ monohydroxyalkyl radical; a C₂-C₄ polyhydroxyalkyl radical; a C₁-C₄ monohydroxyalkoxy radical; a C₂-C₄ polyhydroxyalkoxy radical; a C₁-C₄ aminoalkoxy radical; an amino radical monosubstituted or disubstituted with a C₁-C₄ alkyl radical, C₁-C₄ monohydroxyalkyl radical, C₂-C₄ polyhydroxyalkyl radical, C₁-C₄ aminoalkyl radical, mono(C₁-C₄)alkylamino(C₁-C₄)alkyl radical, di(C₁-C₄)alkylamino(C₁-C₄)alkyl radical, C₁-C₄ ureidoalkyl radical, aryl radical, or aryl radical in which the aryl ring is substituted with one or more hydroxyl, carboxyl, amino or di(C₁-C₄)alkylamino radicals,
R20 represents a hydrogen or halogen atom, a C₁-C₄ alkyl radical or a nitro group.

Among the nitrobenzene dyes of formula (X) above, mention may be made most particularly of: 2-amino-4-methyl-5-N-(β-hydroxyethyl)aminonitrobenzene; 4-N-(β-ureidoethyl)aminonitrobenzene; 4-(N-ethyl-N-β-hydroxyethyl)amino-1-N-(β-hydroxyethyl)aminonitrobenzene; 2-N-(β-hydroxyethyl)amino-5-methylnitrobenzene; 5-chloro-3-N-(ethyl)amino-4-hydroxynitrobenzene; 5-amino-3-chloro-4-hydroxynitrobenzene; 2-N-(γ-hydroxypropyl)amino-5-N,N-bis(β-hydroxyethyl)aminonitrobenzene; 5-hydroxy-2-N-(γ-hydroxypropyl)aminonitrobenzene; 1,3-bis(β-hydroxyethyl)amino-4-chloro-6-nitrobenzene; 2,4-diaminonitrobenzene; 3,4-diaminonitrobenzene; 2,5-diaminonitrobenzene; 3-amino-4-hydroxynitrobenzene; 4-amino-3-hydroxynitrobenzene; 5-amino-2-hydroxynitrobenzene; 2-amino-5-hydroxynitrobenzene; 4-amino-3-hydroxynitrobenzene; 5-amino-2-hydroxynitrobenzene; 2-amino-3-hydroxynitrobenzene; 2-amino-5-N-(β-hydroxyethyl)aminonitrobenzene; 2-amino-5-N,N-bis(β-hydroxyethyl)aminonitrobenzene; 2,5-N,N′-(β-hydroxyethyl)aminonitrobenzene; 2-N-(β-hydroxyethyl)amino-5-N,N-bis(β-hydroxyethyl)aminonitrobenzene; 2-amino-5-N-(methyl)aminonitrobenzene; 2-N-(methyl)amino-5-N,N-bis(β-hydroxyethyl)aminonitrobenzene; 2-N-(methyl)amino-5-(N-methyl-N-β-hydroxyethyl)aminonitrobenzene; 2,5-N,N′-(β-hydroxyethyl)aminonitrobenzene; 2-N-(β-hydroxyethyl)amino-5-hydroxynitrobenzene; 3-methoxy-4-N-(β-hydroxyethyl)aminonitrobenzene; 2-N-(methyl)amino-4-β-hydroxyethyloxynitrobenzene; 2-amino-3-methylnitrobenzene; 2-N-(β-hydroxyethyl)amino-5-aminonitrobenzene; 2-amino-4-chloro-5-N-(β-hydroxyethyl)aminonitrobenzene; 2-amino-4-methyl-5-N-(β-hydroxyethyl)aminonitrobenzene; 2-amino-4-methyl-5-N-(methyl)aminonitrobenzene; 2-N-(β-hydroxyethyl)amino-5-methoxynitrobenzene; 2-amino-5-β-hydroxyethyloxynitrobenzene; 2-N-(β-hydroxyethyl)aminonitrobenzene; 3-amino-4-N-(β-hydroxyethyl)aminonitrobenzene; 3-β-hydroxyethyloxy-4-N-(β-hydroxyethyl)aminonitrobenzene; 2-N-(methyl)amino-4-β,γ-dihydroxypropyloxynitrobenzene; 2-N-(β-hydroxyethyl)amino-5-β-hydroxyethyloxynitrobenzene; 2-N-(β-hydroxyethyl)amino-5-β,γ-dihydroxypropyloxynitrobenzene; 2-hydroxy-4-N-(β-hydroxyethyl)aminonitrobenzene; 2-N-(methyl)amino-4-methyl-5-aminonitrobenzene; 2-amino-4-isopropyl-5-N-(methyl)aminonitrobenzene; 2-N-(methyl)amino-5-(N-methyl-N-β,γ-dihydroxypropyl)aminonitrobenzene; 3-N-(β-hydroxyethyl)amino-4-N-(β-hydroxyethyl)aminonitrobenzene; 2-amino-4-methyl-5-N-(β,γ-dihydroxypropyl)aminonitrobenzene; 2-amino-4-methyl-5-hydroxynitrobenzene; 2-N-(β-hydroxyethyl)amino-4-N-(β-hydroxyethyl)aminonitrobenzene; 2-amino-5-N-(β-aminoethyl)aminonitrobenzene; 2-N-(β-aminoethyl)amino-5-methoxynitrobenzene; 2-N-(methyl)amino-5-N-(β-aminoethyl)aminonitrobenzene; 2-N-(β-aminoethyl)amino-4-N,N-(dimethyl)aminonitrobenzene; 3-amino-4-N-(β-aminoethyl)aminonitrobenzene; 2-amino-4-methyl-5-N-(β-aminoethyl)aminonitrobenzene; 2-N-(β-aminoethyl)amino-5-N,N-bis(β-hydroxyethyl)aminonitrobenzene; 3-β-aminoethyloxy-4-aminonitrobenzene; 2-N-(methyl)amino-5-(N-6-amino-n-butyl)aminonitrobenzene; 2-N-(γ-amino-n-propyl)amino-5-N,N-(dimethyl)aminonitrobenzene; 3-methoxy-4-N-(β-aminoethyl)aminonitrobenzene; 2-N-(β-aminoethyl)amino-5-aminonitrobenzene; 2-amino-4-chloro-5-N-(β-aminoethyl)aminonitrobenzene; 2-N-(β-aminoethyl)amino-4-methoxynitrobenzene; 2-N-(β-aminoethyl)aminonitrobenzene; 2-N-(β-aminoethyl)amino-5-N-(β-aminoethyl)aminonitrobenzene; 2-N-(β-aminoethyl)amino-4-β-hydroxyethyloxynitrobenzene; 3-β-hydroxyethyloxy-4-N-(β-aminoethyl)aminonitrobenzene; 2-amino-5-aminoethyloxynitrobenzene; 3-hydroxy-4-N-(β-aminoethyl)aminonitrobenzene; 2-N-(β-aminoethyl)amino-5-β-hydroxyethyloxynitrobenzene; 2-N-(β-aminoethyl)amino-4-hydroxynitrobenzene; 2-[2-hydroxy-3-N-(β-hydroxyethyl)amino-6-nitro]benzyloxy]ethylamine and 2-[2-hydroxy-3-N-(β-hydroxypropyl)amino-6-nitro]benzyloxy]ethylamine.

Among the nitrobenzene dyes of formula (X) above, the following are most particularly preferred: 2-amino-4-methyl-5-N-(β-hydroxyethyl)aminonitrobenzene; 4-N-(β-ureidoethyl)aminonitrobenzene; 4-(N-ethyl-N-β-hydroxyethyl)amino-1-N-(β-hydroxyethyl)aminonitrobenzene; 2-N-(β-hydroxyethyl)amino-5-methylnitrobenzene; 5-chloro-3-N-(ethyl)amino-4-hydroxynitrobenzene; 5-amino-3-chloro-4-hydroxynitrobenzene; 2-N-(γ-hydroxypropyl)amino-5-N,N-bis(3-hydroxyethyl)aminonitrobenzene; 5-hydroxy-2-N-(γ-hydroxypropyl)aminonitrobenzene; 1,3-bis(j3-hydroxyethyl)amino-4-chloro-6-nitrobenzene; 3,4-diaminonitrobenzene; 2-amino-5-hydroxynitrobenzene; 2-amino-3-hydroxynitrobenzene; 2-amino-5-N-(β-hydroxyethyl)aminonitrobenzene; 2-amino-5-N,N-bis(O-hydroxyethyl)aminonitrobenzene; 2-N-(β-hydroxyethyl)amino-5-N,N-bis(o-hydroxyethyl)aminonitrobenzene; 2-N-(β-hydroxyethyl)amino-5-hydroxynitrobenzene; 2-N-(β-hydroxyethyl)amino-5-aminonitrobenzene; 2-N-(β-aminoethyl)amino-4-methoxynitrobenzene; and 2-N-(β-aminoethyl)amino-5-β-hydroxyethyloxynitrobenzene.

According to a particular embodiment, the direct dye(s) are chosen from porphyrins, and phthalocyanines, alone or as mixtures.

According to one particular embodiment, the direct dye(s) of the invention are cationic.

Examples of suitable direct dyes that may be mentioned include azo direct dyes; (poly)methine dyes such as cyanines, hemicyanines and styryl dyes; carbonyl dyes; azine dyes; nitro(hetero)aryl dyes; tri(hetero)arylmethane dyes; porphyrin dyes; phthalocyanine dyes, and natural direct dyes, alone or as mixtures.

Preferably, the direct dye(s) contain at least one quaternized cationic chromophore or at least one chromophore bearing a quaternized or quaternizable cationic group.

According to a particular embodiment of the invention, the direct dyes comprise at least one quaternized cationic chromophore.

Mention may in particular be made, for the cationic azo dyes, of those resulting from the cationic dyes described in the Kirk-Othmer Encyclopedia of Chemical Technology, “Dyes, Azo”, J. Wiley & Sons, updated on 19 Apr. 2010.

Among the azo dyes that may be used according to the invention, mention may be made of the cationic azo dyes described in patent applications WO 95/15144, WO 95/01772 and EP-714954.

According to a preferred embodiment of the invention, the direct dye(s) are chosen from cationic dyes known as “basic dyes”.

Among the azo dyes described in the Colour Index International 3rd edition, mention may be made in particular of the following compounds: Basic Red 22, Basic Red 76, Basic Yellow 57, Basic Brown 16 and Basic Brown 17.

Among the cationic quinone dyes, those mentioned in the abovementioned Colour Index International are suitable and, among these, mention may be made, inter alia, of the following dyes: Basic Blue 22 and Basic Blue 99.

Among the azine dyes that are suitable, mention may be made of those listed in the Colour Index International, for example the following dyes: Basic Blue 17, Basic Red 2.

Among the cationic triarylmethane dyes that may be used according to the invention, mention may be made, in addition to those listed in the Colour Index, of the following dyes: Basic Green 1, Basic Violet 3, Basic Violet 14, Basic Blue 7 and Basic Blue 26.

Mention may also be made of the cationic dyes in U.S. Pat. No. 5,888,252, EP 1 133 975, WO 03/029 359, EP 860 636, WO 95/01772, WO 95/15144 and EP 714 954. Mention may also be made of those listed in the encyclopaedia “The Chemistry of Synthetic Dyes” by K. Venkataraman, 1952, Academic Press, vol. 1 to 7, in the “Kirk-Othmer Encyclopedia of Chemical Technology”, chapter “Dyes and Dye Intermediates”, 1993, Wiley and Sons, and in various chapters of “Ullmann's Encyclopedia of Industrial Chemistry”, 7th edition, Wiley and Sons.

Preferably, the cationic direct dyes are chosen from those resulting from dyes of azo and hydrazono type.

According to a particular embodiment, the direct dyes are cationic azo dyes, described in EP 850 636, FR 2 788 433, EP 920 856, WO 99/48465, FR 2 757 385, EP 850 637, EP 918 053, WO 97/44004, FR 2 570 946, FR 2 285 851, DE 2 538 363, FR 2 189 006, FR 1 560 664, FR 1 540 423, FR 1 567 219, FR 1 516 943, FR 1 221 122, DE 4 220 388, DE 4 137 005, WO 01/66646, U.S. Pat. No. 5,708,151, WO 95/01772, WO 515 144, GB 1 195 386, U.S. Pat. Nos. 3,524,842, 5,879,413, EP 1 062 940, EP 1 133 976, GB 738 585, DE 2 527 638, FR 2 275 462, GB 1974-27645, Acta Histochem. (1978), 61(1), 48-52; Tsitologiya (1968), 10(3), 403-5; Zh. Obshch. Khim. (1970), 40(1), 195-202; Ann. Chim. (Rome) (1975), 65(5-6), 305-14; J. Chinese Chem. Soc. (Taipei) (1998), 45(1), 209-211; Rev. Roum. Chim. (1988), 33(4), 377-83; Text. Res. J. (1984), 54(2), 105-7; Chim. Ind. (Milan) (1974), 56(9), 600-3; Khim. Tekhnol. (1979), 22(5), 548-53; Ger. Monatsh. Chem. (1975), 106(3), 643-8; MRL Bull. Res. Dev. (1992), 6(2), 21-7; Lihua Jianyan, Huaxue Fence (1993), 29(4), 233-4; Dyes Pigm. (1992), 19(1), 69-79; Dyes Pigm. (1989), 11(3), 163-72.

Preferably, the cationic direct dye(s) comprise a quaternary ammonium group; more preferably, the cationic charge is endocyclic.

These cationic radicals are, for example, a cationic radical:
bearing an exocyclic (di/tri)(C₁-C₈)alkylammonium charge, or
bearing an endocyclic charge, such as comprising a cationic heteroaryl group chosen from: acridinium, benzimidazolium, benzobistriazolium, benzopyrazolium, benzopyridazinium, benzoquinolium, benzothiazolium, benzotriazolium, benzoxazolium, bipyridinium, bis-tetrazolium, dihydrothiazolium, imidazopyridinium, imidazolium, indolium, isoquinolium, naphthoimidazolium, naphthooxazolium, naphthopyrazolium, oxadiazolium, oxazolium, oxazolopyridinium, oxonium, phenazinium, phenooxazolium, pyrazinium, pyrazolium, pyrazoyltriazolium, pyridinium, pyridinoimidazolium, pyrrolium, pyrylium, quinolium, tetrazolium, thiadiazolium, thiazolium, thiazolopyridinium, thiazoylimidazolium, thiopyrylium, triazolium or xanthylium.

Mention may be made of the hydrazono cationic dyes of formulae (XII) and (XIII), and the azo cationic dyes of formulae (XIV) and (XV) below:

TABLE 3

| | |
|---|---|
| Het⁺—C(Ra)═N—N(Rb)—Ar, Q⁻ | Het⁺—N(Ra)—N═C(Rb)—Ar, Q⁻ |
| (XII) | (XIII) |
| Het⁺—N═N—Ar, Q⁻ | Ar⁺—N═N—Ar″, Q⁻ |
| (XIV) | (XV) |

in which formulae (XII) to (XV):
Het⁺ represents a cationic heteroaryl radical, preferably bearing an endocyclic cationic charge, such as imidazolium, indolium or pyridinium, optionally substituted, preferably with at least one (C₁-C₈) alkyl group such as methyl;
Ar⁺ represents an aryl radical, such as phenyl or naphthyl, bearing an exocyclic cationic charge, preferably tri(C₁-C₈)alkylammonium such as trimethylammonium;
Ar represents an aryl group, especially phenyl, which is optionally substituted, preferably with one or more electron-donating groups such as i) optionally substituted (C₁-C₈)alkyl, ii) optionally substituted (C₁-C₈)alkoxy, iii) (di)(C₁-C₈)(alkyl)amino optionally substituted on the alkyl group(s) with a hydroxyl group, iv) aryl(C₁-C₈)alkylamino, v) optionally substituted N—(C₁-C₈)alkyl-N-aryl(C₁-C₈)alkylamino or alternatively Ar represents a julolidine group;
Ar″ represents an optionally substituted (hetero)aryl group such as phenyl or pyrazolyl, which are optionally substituted, preferably by one or more (C₁-C₈)alkyl, hydroxyl, (di)(C₁-C₈)(alkyl)amino, (C₁-C₈)alkoxy or phenyl groups;
Ra and Rb, which may be identical or different, represent a hydrogen atom or a (C₁-C₈)alkyl group, which is optionally substituted, preferably with a hydroxyl group;
or else the substituent Ra with a substituent of Het⁺ and/or Rb with a substituent of Ar form, together with the atoms that bear them, a (hetero)cycloalkyl; in particular, Ra and Rb represent a hydrogen atom or a (C₁-C₄)alkyl group optionally substituted with a hydroxyl group;
Q⁻ represents an organic or mineral anionic counterion, such as a halide or an alkyl sulfate.

In particular, mention may be made of the azo and hydrazono direct dyes bearing an endocyclic cationic charge of formulae (XII) to (XV) as defined previously, more particularly the cationic direct dyes of formulae (XII) to (XV) bearing an endocyclic cationic charge, described in patent applications WO 95/15144, WO 95/01772 and EP 714 954, preferably the following direct dyes:

TABLE 4

| | |
|---|---|
| 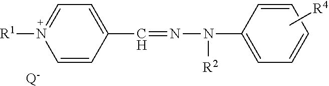 | (XII-1) |
| 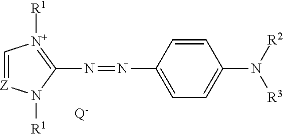 | (XIV-1) |

in which formulae (XII-1) and (XIV-1):
R1 represents a (C₁-C₄)alkyl group such as methyl;
R2 and R3, which may be identical or different, represent a hydrogen atom or a (C₁-C₄)alkyl group such as methyl; and
R4 represents a hydrogen atom or an electron-donating group such as optionally substituted (C₁-C₈)alkyl, optionally substituted (C₁-C₈)alkoxy, or (di)(C₁-C₈)(alkyl)amino optionally substituted on the alkyl group(s) with a hydroxyl group; particularly, R4 is a hydrogen atom,
Z represents a CH group or a nitrogen atom, preferably CH,
   Q⁻ is an anionic counterion as defined previously, in particular a halide, such as chloride, or an alkyl sulfate, such as methyl sulfate or mesityl.

Particularly, the dyes of formulae (XII-1) and (XIV-1) are chosen from Basic Red 51, Basic Yellow 87 and Basic Orange 31 or derivatives thereof:

TABLE 5

| | |
|---|---|
| 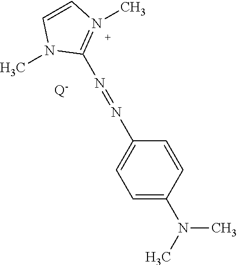 | Basic Red 51 |
| 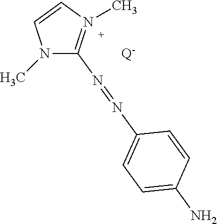 | Basic Orange 31 |
| 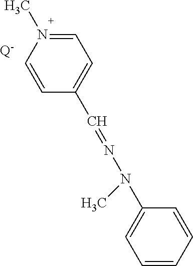 | Basic Yellow 87 |

with Q⁻ being an anionic counterion as defined previously, in particular a halide, such as chloride, or an alkyl sulfate, such as methyl sulfate or mesityl.

According to a particular embodiment of the invention, the direct dyes are fluorescent, i.e. they comprise at least one fluorescent chromophore as defined above.

Fluorescent dyes that may be mentioned include the radicals resulting from the following dyes: acridines, acridones, benzanthrones, benzimidazoles, benzimidazolones, benzindoles, benzoxazoles, benzopyrans, benzothiazoles, coumarins, difluoro{2-[(2H-pyrrol-2-ylidene-kN)methyl]-1H-pyrrolato-kN}borons (BODIPY®), diketopyrrolopyrroles, fluorindines, (poly)methines (notably cyanines and styryls/hemicyanines), naphthalimides, naphthanilides, naphthylamines (such as dansyls), oxadiazoles, oxazines, perilones, perinones, perylenes, polyenes/carotenoids, squaranes, stilbenes and xanthenes.

Mention may also be made of the fluorescent dyes described in EP 1 133 975, WO 03/029 359, EP 860 636, WO 95/01772, WO 95/15144 and EP 714 954 and those listed in the encyclopaedia “The Chemistry of Synthetic Dyes” by K. Venkataraman, 1952, Academic Press, vol. 1 to 7, in the “Kirk-Othmer Encyclopedia of Chemical Technology”, in the chapter “Dyes and Dye Intermediates”, 1993, Wiley and Sons, and in various chapters of “Ullmann's Encyclopedia of Industrial Chemistry”, 7th edition, Wiley and Sons, and in the handbook—“A Guide to Fluorescent Probes and Labeling Technologies”, 10th Ed., Molecular Probes/Invitrogen—Oregon 2005, circulated on the Internet or in the preceding printed editions.

According to a preferred variant of the invention, the fluorescent dye(s) are cationic and comprise at least one quaternary ammonium radical, such as those of formula (XV) below:

W⁺—[C(Rc)═C(Rd)]_{m′}-Ar,Q⁻ (XV)

in which formula (XV):
W⁺ represents a cationic heterocyclic or heteroaryl group, particularly comprising a quaternary ammonium optionally substituted with one or more (C₁-C₈)alkyl groups optionally substituted especially with one or more hydroxyl groups;
Ar representing an aryl group such as phenyl or naphthyl, optionally substituted preferably with i) one or more halogen atoms such as chlorine or fluorine; ii) one or more (C₁-C₈)alkyl groups, preferably of C₁-C₄ such as methyl; iii) one or more hydroxyl groups; iv) one or more (C₁-C₈)alkoxy groups such as methoxy; v) one or more hydroxy(C₁-C₈)alkyl groups such as hydroxyethyl, vi) one or more amino groups or (di)(C₁-C₈)alkylamino, preferably with the C₁-C₄ alkyl part optionally substituted with one or more hydroxyl groups, such as (di)hydroxyethylamino, vii) with one or more acylamino groups; viii) one or more heterocycloalkyl groups such as piperazinyl, piperidyl or 5- or 6-membered heteroaryl such as pyrrolidinyl, pyridyl and imidazolinyl;
m′ represents an integer between 1 and 4 inclusive, and in particular m is 1 or 2; more preferably 1;
Rc and Rd, which may be identical or different, represent a hydrogen atom or an optionally substituted (C₁-C₈)alkyl group, preferably of C₁-C₄, or alternatively Rc contiguous with Mi⁺ and/or Rd contiguous with Ar form, with the atoms that bear them, a (hetero)cycloalkyl, particularly Rc is contiguous with W⁺ and form a (hetero)cycloalkyl such as cyclohexyl;
Q⁻ is an organic or mineral anionic counterion as defined above.

According to one particular embodiment of the invention, the direct dyes are natural.

The expression “natural dye” is intended to mean any dye or dye precursor that has at least one natural occurrence and that is produced by extraction (and optionally purification) from a plant matrix, or via milling. Natural dyes may also be obtained by fermentation.

The natural dye(s) are chosen, for example, from lawsone, juglone, alizarin, purpurin, carminic acid, kermesic acid, laccaic acid, purpurogallin, anthragallol, protocatechaldehyde, indigo, isatin, curcumin, spinulosin, chlorophylls, chlorophyllines, orceins, haematein, haematoxylin, brazilin, brazileine, santaline, santarubin, carthamine, flavonoids (with, for example, morin, apigenidin and quercetin), anthocyans (such as apigeninidin) and carotenoids, or mixtures thereof.

Use may also be made of the extracts, decoctions or milled materials (such as powders) containing these natural dyes, and in particular the extracts, decoctions or milled materials obtained for example from henna, pernambuco wood, logwood, sandalwood, orchil, turmeric, madder, true indigo, sorghum, cochineal, carrots, annatto, murex, Brazil wood, safflower.

Preferably, the natural dye(s) are chosen from lawsone, juglone, alizarin, purpurin, carminic acid, kermesic acid, laccaic acid, protocatechaldehyde, indigo, isatin, curcumin, spinulosin, apigenidin, chlorophylline, sorghum extracts, orceins, cochineal carmine, haematein, haematoxylin, brazilin, brazileine, extracts of logwood wood, cudbear wood and Brazil wood, and mixtures thereof.

The chemical oxidizing agent(s) may be chosen from hydrogen peroxide, urea peroxide, alkali metal bromates or ferricyanides, peroxygenated salts, for instance alkali metal or alkaline-earth metal persulfates, perborates and percarbonates, and also peracids and precursors thereof.

Preferably, the chemical oxidizing agent(s) are chosen from solid chemical oxidizing agents, and more preferably from urea peroxide, alkali metal bromates or ferricyanides, or peroxygenated salts, for instance alkali metal or alkaline-earth metal persulfates, perborates and percarbonates.

Better still, the oxidizing agent(s) are chosen from peroxygenated salts and even more preferably from persulfates.

In accordance with the present invention, the substrate is pretreated with a dyeing or lightening composition, termed dyeing or lightening preparation composition, comprising the dye(s) chosen from oxidation dyes and/or direct dyes, more preferably oxidation dyes, and/or oxidizing agent(s).

Thus, the dyeing or lightening composition may cover all or part of the surface of the substrate. The surface of the substrate may thus be entirely or partially covered with one or more layers forming a dyeing or lightening composition.

Preferably, the dyeing or lightening preparation composition is deposited on part of the surface of the substrate so as to represent patterns, which, after contact with the keratin fibres, will make it possible to produce the coloured or bleached patterns on said fibres. In other words, the dye(s) or the oxidizing agent(s) are deposited in the form of patterns on the surface of the substrate.

Thus, the surface of the substrate includes one or more layers constituted of the dyeing or lightening composition arranged in one or more particular geometric shapes, known as patterns, which, after reaction, lead to the production of coloured or bleached patterns on said fibres.

In other words, the surface of the substrate may comprise, along a longitudinal axis, one or more layers comprising one or more dyes, preferably oxidation dyes, or one or more oxidizing agents having varied shapes, in particular geometric shapes, so as to form patterns.

The pattern(s) may thus have any shape, in particular a geometric shape.

Preferably, the patterns may be squares, circles, ovals, ellipses or triangles, in the form of filled patterns or of lines surrounding these patterns. They may also be thick or thin, straight or curved lines, crossed lines, representing letters, stylized drawings or geometric patterns. They may also be dotted lines or spots.

The substrate may comprise a copy of the desired pattern(s) on the face opposite the face bearing the dye(s) or the oxidizing agent(s). The production of these patterns on the opposite face makes it possible to indicate the place where the dye(s) or the oxidizing agent(s) may then be deposited on the surface of the substrate. Such a production facilitates thereafter the emplacement of the substrate on the keratin fibres at the place where it is the aim to produce the pattern. In other words, the presence of the patterns on the opposite face makes it possible to indicate the place where the dyeing or lightening preparation composition may be deposited.

According to one preferred embodiment, the substrate comprises a surface coated with at least one layer containing one or more oxidation dyes or one or more chemical oxidizing agents.

In accordance with this embodiment, the oxidation dyes are chosen from couplers and more preferably heterocyclic or benzene-based couplers with at least one amine substituent on the aromatic ring.

In accordance with this embodiment, the substrate is preferably an element in sheet form, preferably a plastic sheet.

In accordance with this embodiment, the substrate comprises a surface comprising, along a longitudinal axis, several layers containing one or more oxidation dyes having varied shapes, in particular geometric shapes.

According to one preferred embodiment, the substrate comprises a surface on which is printed at least one layer containing one or more oxidation dyes or one or more chemical oxidizing agents.

Preferably, the substrate comprises a surface on which is printed at least one layer in the form of one or more patterns containing one or more oxidation dyes or one or more chemical oxidizing agents.

Preparation of the Substrate

Preferably, the substrate is pretreated with at least one dyeing preparation composition comprising one or more dyes as defined above or with a lightening composition comprising one or more oxidizing agents.

The dyeing or lightening preparation composition may be liquid or in pulverulent form at ambient temperature, preferably liquid at ambient temperature.

The dyeing or lightening preparation composition preferably comprises one or more alkaline agents as defined above.

When said dyeing or lightening preparation composition is aqueous and contains one or more alkaline agents, the pH of said composition preferably ranges from 7.5 to 13, better still from 8 to 12 and even better still from 8 to 11.

Preferably, the process for manufacturing the substrate as defined previously comprises at least one step of depositing onto the surface of said substrate at least one dyeing preparation composition containing one or more dyes or a lightening composition comprising one or more oxidizing agents, and at least one step of partially or totally drying said substrate; more preferably, said dyeing or lightening preparation composition is deposited onto the surface of the substrate by means of a printing method.

In other words, the dyeing or lightening preparation composition(s) are printed on the surface of a substrate, that is to say using a printing process which makes it possible to obtain the substrate defined previously.

According to this preference, the printing method which serves to deposit the dyeing or lightening composition(s) onto the surface of the substrate may be a screen printing process, a flexography process, an offset printing process, an inkjet printing process or a laser printing process.

Preferably, the dyeing or lightening preparation composition is printed onto the surface of the substrate by means of an inkjet printing process or a laser printing process, notably using an inkjet printer or a laser printer.

This preferred manufacturing process, corresponding to a process for printing the substrate, may be performed in the hairstyling salon itself, notably by means of the presence of an inkjet printer or a laser printer, before performing the dyeing or lightening process according to the invention.

Alternatively, this preferred process may also be performed outside the hairstyling salon and as such the user merely has to use the substrates to dye the hair.

In this case, the pretreated substrate may be supplied to the user to produce a unified colouring and/or patterns on the hair.

Preferably, the dyeing preparation composition containing the dye(s) or the lightening preparation composition is deposited in the form of one or more patterns onto the surface of a substrate. In particular, the patterns may be squares, circles, ovals, ellipses or triangles, in the form of filled patterns or of lines surrounding these patterns. They may also be thick or thin, straight or curved lines, crossed lines, representing letters, stylized drawings or geometric patterns. They may also be dotted lines or spots.

Once the dyeing or lightening preparation composition(s) have been deposited at the surface of the substrate, the substrate is then dried.

The substrate thus pretreated in accordance with the manufacturing process preferably dries within a period ranging from 5 minutes to 120 minutes, preferably from 5 minutes to 90 minutes, more preferably from 1 minute to 60 minutes and better still from 5 minutes to 60 minutes.

Preferably, the step of drying said substrate consists in leaving said substrate to dry in the open air.

Once the substrate has been prepared, it comprises a surface coated with at least one layer comprising one or more dyes as defined above, preferably oxidation dyes, or one or more oxidizing agents.

The layer coating the surface of the substrate is a dyeing composition comprising one or more dyes as defined above, preferably oxidation dyes.

In accordance with the present invention, the layer preferably comprises a total water content of less than 20% by weight, preferably less than or equal to 15% by weight, more preferably less than or equal to 10% by weight, relative to the total weight of the dyeing composition.

Additional Step(s) of the Process

According to one embodiment, the process according to the invention may also comprise the use of a composition (B) free of hydrogen peroxide after rinsing of the aqueous composition or after the use of the hand-held styling device.

Composition (B) may comprise one or more organic solvents as described previously, preferably chosen from polyols containing more than two hydroxyl functions, such as glycerol.

Preferably, composition (B) may be aqueous and may comprise one or more organic solvents.

Composition (B) may also further comprise one or more surfactants, preferably one or more non-ionic and/or anionic surfactants.

Preferably, the non-ionic surfactants are oxyalkylenated and chosen from oxyethylenated C₈-C₃₀ alcohols, and polyoxyethylenated esters of saturated or unsaturated, linear or branched C₈-C₃₀ acids and of sorbitol.

Composition (B) may also further comprise one or more fatty substances.

The term “fatty substance” is intended to mean an organic compound that is insoluble in water at ordinary temperature (25° C.) and at atmospheric pressure (760 mmHg) (solubility of less than 5%, preferably less than 1% and even more preferably less than 0.1%). They bear in their structure at least one hydrocarbon-based chain including at least 6 carbon atoms or a sequence of at least two siloxane groups. In addition, the fatty substances are generally soluble in organic solvents under the same temperature and pressure conditions, for instance chloroform, ethanol, benzene, liquid petroleum jelly or decamethylcyclopentasiloxane.

According to the invention, the fatty substances are chosen from compounds that are liquid or pasty at ambient temperature and at atmospheric pressure.

More particularly, the fatty substance(s) are chosen from C₆-C₁₆ lower alkanes, non-silicone oils of animal, plant, mineral or synthetic origin, fatty alcohols, esters of a fatty acid and/or of a fatty alcohol, non-silicone waxes and silicones.

It is recalled that, for the purposes of the invention, fatty alcohols, esters and acids more particularly bear at least one linear or branched, saturated or unsaturated hydrocarbon-based group comprising 6 to 30 carbon atoms, which is optionally substituted, in particular with one or more hydroxyl groups (in particular 1 to 4). If they are unsaturated, these compounds may comprise one to three conjugated or unconjugated carbon-carbon double bonds.

Preferably, the fatty substance(s) are chosen from fatty alcohols, notably those chosen from linear or branched, saturated or unsaturated alcohols including from 8 to 30 carbon atoms. Mention may be made, for example, of cetyl alcohol, stearyl alcohol and a mixture thereof (cetylstearyl alcohol), octyldodecanol, 2-butyloctanol, 2-hexyldecanol, 2-undecylpentadecanol, oleyl alcohol or linoleyl alcohol.

More preferably, composition (B) comprises cetyl alcohol, stearyl alcohol and a mixture thereof, in particular cetyl alcohol.

The aqueous composition and/or composition (B) may comprise one or more adjuvants such as anionic, cationic, non-ionic, amphoteric or zwitterionic polymers or mixtures thereof, mineral thickeners, and in particular fillers such as clays or talc, organic thickeners/gelling agents with, in particular, anionic, cationic, non-ionic and amphoteric polymeric associative thickeners other than the polymers mentioned previously, antioxidants, penetrants, sequestrants, fragrances, dispersants, film-forming agents, ceramides, preserving agents, opacifiers.

The adjuvants may be present for each of them in an amount ranging from 0.01% to 20% by weight relative to the total weight of the composition under consideration.

Assembly Intended for Dyeing or Lightening

As indicated above, a subject of the invention is also an assembly intended for dyeing and/or lightening keratin fibres, in particular human keratin fibres such as the hair, comprising at least one aqueous composition, as defined above, and at least one hand-held styling device as defined above.

Preferably, the assembly also comprises:
a computing device configured for calculating the number of uses of the substrate(s) on the keratin fibres,
a styling device as defined above, in which the first jaw and/or the second jaw also comprise one or more means for detecting the moving of the jaws into an open position and/or the moving of the jaws into a closed position; said detection means being connected to the computing device.

The assembly makes it possible in particular to indicate to the user the number of times that one and the same substrate has been used to produce one or more coloured or bleached patterns and/or the number of times that one and the same substrate can still be used before total extraction of the dyes.

The computing device can thus be configured for emitting a sound signal indicating the number of times that one and the same substrate has been used to produce one or more coloured or bleached patterns and/or the number of times that one and the same substrate can still be used before total extraction of the dyes.

Preferably, the computing device can also be connected to an image acquisition device configured for visualizing on a screen the surface of the substrate(s) used on the keratin fibres.

By virtue of the image acquisition device, the assembly helps in particular the user identify and visualise the pattern to be placed on the keratin fibres, the number of applications of the pattern(s) and/or the patterns remaining unused.

The image acquisition device is particularly advantageous when the substrate(s) is or are mounted on at least one treatment surface of the styling device, in particular the treatment surface intended to be heated, by means of one or more dispensing means as described above.

In this case, the dispensing means is or are connected to the computing device, in particular by means of the feed drum.

The computing device can also comprise a control member configured for decreasing the heating temperature of the treatment surface intended to be heated of the hand-held styling device.

Preferably, the detection means are therefore also connected to the member for controlling the computing device.

In this way, the member for controlling the computing device can cut the heating temperature of the treatment surface intended to be heated after application of the substrate to the keratin fibres. The detection means connected to the control member make it possible to protect the integrity of the dyes against the heat and to improve the lifetime of the substrates.

Preferably, the detection means are chosen from infrared or thermocouple sensors.

The computing device may also comprise at least one piece of software configured for recording and loading the dyeing or lightening patterns that it is the aim to produce on the keratin fibres, in particular at various places on the head of hair.

By virtue of this embodiment, users can record and load, optionally remotely, in the hair salon of their choice, the type of coloured or bleached patterns, in particular their geometric shape or their colour, and also the number of coloured or bleached patterns that they wish to produce on a head of hair.

Thus, the user can programme, optionally remotely, by means of a human-machine interface, such as for example a touchscreen tablet, connected to the computing device, the type and number of coloured or bleached patterns to be produced.

Further aims, features and advantages of the invention will become apparent from reading the following description, which is given only by way of non-limiting example and with reference to the appended drawings, in which:

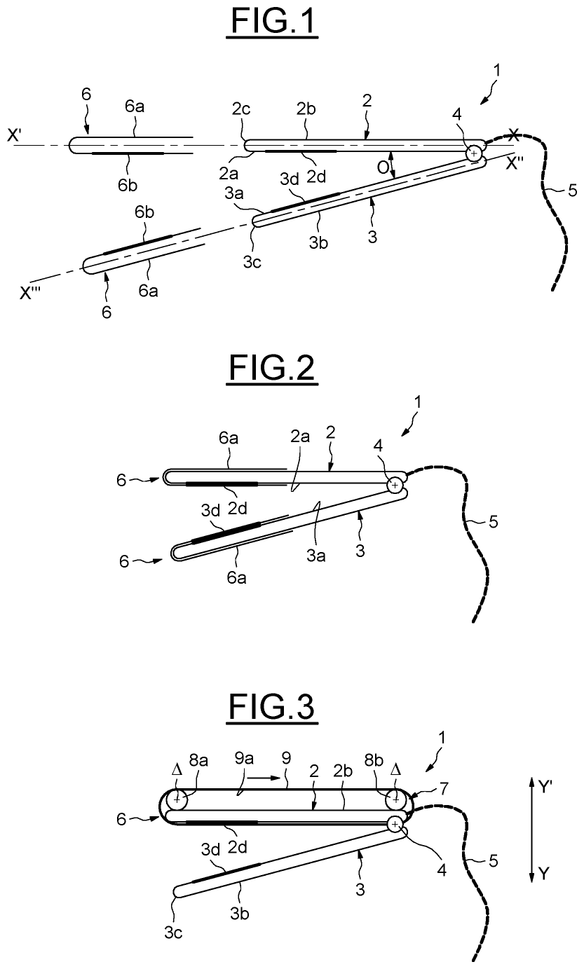

FIG. 1 diagrammatically represents a longitudinal sectional view of a hand-held styling device according to the invention,

FIG. 2 diagrammatically represents a longitudinal sectional view of the hand-held styling device of FIG. 1, with substrates according to the invention,

FIG. 3 diagrammatically represents a longitudinal sectional view of the hand-held styling device of FIG. 1 with a substrate mounted mobile on the first jaw by means of pivoting dispensing means according to a first embodiment,

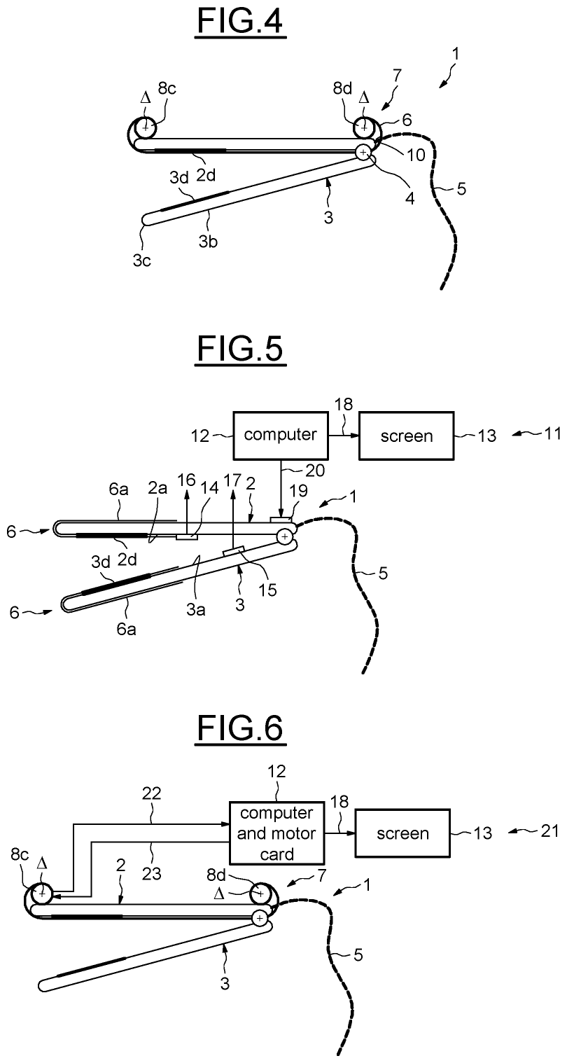

FIG. 4 diagrammatically represents a longitudinal sectional view of the hand-held styling device of FIG. 1 with a substrate mounted mobile on the first jaw by means of pivoting dispensing means according to a second embodiment,

FIG. 5 diagrammatically represents a longitudinal sectional view of an assembly comprising the hand-held styling device of FIG. 1, a computing device and an image acquisition device,

FIG. 6 diagrammatically represents a longitudinal sectional view of an assembly comprising the hand-held styling device of FIG. 4, a computing device and an image acquisition device,

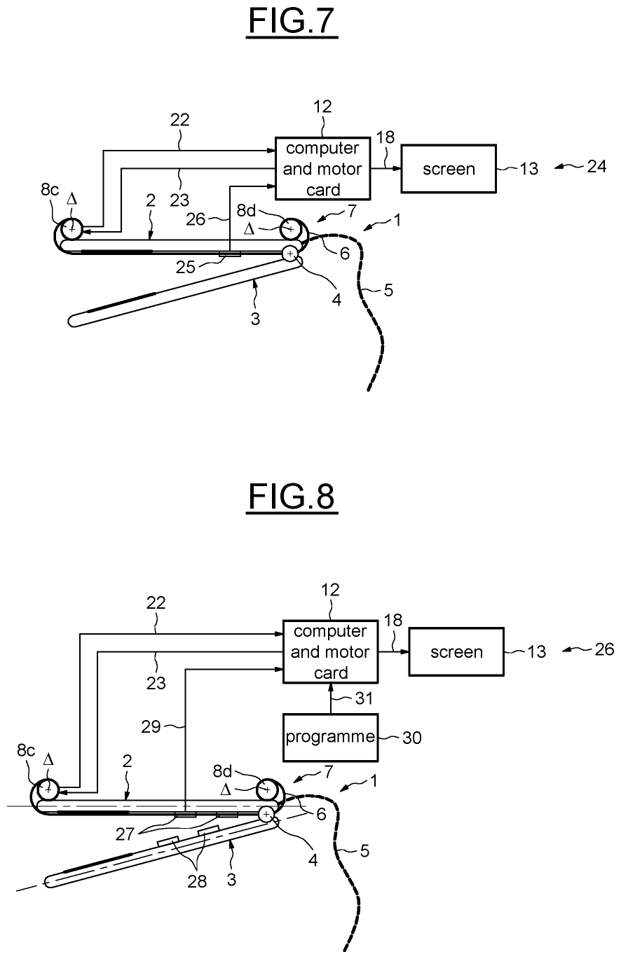

FIG. 7 diagrammatically represents a longitudinal sectional view of an assembly comprising the hand-held styling device of FIG. 4, with a means for closing the jaws, a computing device and an image acquisition device,

FIG. 8 diagrammatically represents a longitudinal sectional view of an assembly comprising the hand-held styling device of FIG. 4, with a means for closing and opening the jaws, a computing device and an image acquisition device.

Represented diagrammatically in FIG. 1 is a hand-held styling device 1 comprising a first jaw 2 which extends along a longitudinal axis X-X′ and has a parallelepipedal geometric shape.

The first jaw 2 has an internal surface 2a, an external surface 2b and an edge 2c which can be at a right angle, as represented in FIG. 1, or can be rounded.

The hand-held styling device 1 also comprises a second jaw 3 which extends along a longitudinal axis X″—X′″ and has a parallelepipedal geometric shape.

The second jaw 3 also has an internal surface 3a, an external surface 3b and an edge 3c which can be at a right angle, as represented in FIG. 1, or can be rounded.

The jaws 2 and 3 are arranged opposite one another and are connected to one another by a hinge 4. Thus, the jaws 2 and 3 are articulated so as to go from an open position O, as represented in FIG. 1, to a closed position F which is not represented.

In FIG. 1, the jaws 2 and 3 have been represented with an identical parallelepipedal geometric shape.

As a variant, the jaws 2 and 3 can have any geometric shape, for example with a curved shape, or a triangular or polyhexagonal shape.

As a variant, the jaws 2 and 3 can have different geometric shapes, which are in particular complimentary to one another, to be intolerable.

The hand-held styling device 1 comprises a first treatment surface 2d borne by the first jaw 2, in particular on the internal surface 2a, and a second treatment surface 3d borne by the second jaw 3, in particular on the internal surface 3a.

In FIG. 1, the first treatment surface 2d and the second treatment surface 3d are respectively located in the vicinity of the free end of the first jaw 2 and of the second jaw 3, in particular the edges 2c and 3c.

The treatment surfaces 2d and 3d extend longitudinally over a portion of the length of the first jaw 2 and of the second jaw 3, respectively. In FIG. 1, the two treatment surfaces 2d and 3d are positioned opposite one another.

The two treatment surfaces 2d and 3d can be brought into contact with the keratin fibres and are suitable for gripping (or pressing) said fibres when they are in the closed position F not represented.

More specifically, in the open position O, as represented in FIG. 1, the keratin fibres are inserted between the jaws 2 and 3, then in the closed position F, the two treatment surfaces 2d and 3d grip said fibres by exerting a pressure. The pressure is maintained for the treatment time and can be actuated manually or automatically.

At least one of the treatment surfaces 2d and 3d is suitable for being heated for the purpose of increasing the temperature of the keratin fibres, in particular of the locks of hair, in a temperature range extending from 60 to 250° C., preferably at a temperature extending from 80 to 180° C., preferably extending from 100° C. to 160° C. and better still from 120 to 150° C. The heating of one of the treatment surfaces 2d and 3d can be carried out by means of one or more heating means, not represented in FIG. 1, which can be activated by a control button located on at least one of the jaws 2 and 3.

Preferably, the two treatment surfaces 2d and 3d are suitable for being heated.

As a variant, one of the treatment surfaces 2d or 3d is suitable for being heated, the other treatment surface 2d or 3d being suitable for bringing the keratin fibres to the corresponding heating treatment surface 2d or 3d when they are in the closed position F not represented.

In FIG. 1, a power cord 5 connects the hand-held styling device 1 to an electric power supply which serves in particular to fix a temperature at at least one of the treatment surfaces 2d or 3d.

In FIG. 1, two substrates, having a general reference 6, comprising a surface 6a coated with at least one layer 6b are represented in the process of being installed at the free ends of the jaws 2 and 3 so as to cover the treatment surfaces 2d and 3d.

According to FIG. 1, the two substrates 6 are thus mounted around the treatment surface 2d and the treatment surface 3d.

In accordance with the embodiment represented in FIG. 1, the two substrates 6 are mounted on the hand-held styling device 1 so as to cover on both sides the treatment surface 2a of the first jaw 2 and the treatment surface 3a of the second jaw 3. As a result, the substrates 6 are mounted on the hand-held styling device 1 so as to partially cover, in particular surround, the internal surface 2a and the external surface 2b of the first jaw 2 and also the internal surface 3a and the external surface 3b of the second jaw 3.

More specifically, according to this embodiment, the two substrates 6 have an oblong, in particular cylindrical, geometric shape, which makes it possible to insert the free end of the first jaw 2 bearing the treatment surface 2d and the free end of the second jaw 3 bearing the treatment surface 3d inside the substrates 6.

Thus, the substrates 6 can be easily installed on the treatment surfaces 2d and 3d of the hand-held styling device 1 in order to produce a colouring, in particular one or more coloured or bleached patterns, during the use on the keratin fibres.

The substrates 6 may be identical or different, that is to say may have at their surface 6a layers comprising dyes, preferably oxidation dyes, or oxidizing agents, having different geometric shapes, in particular different patterns.

As a variant, the substrate 6 may be mounted on the hand-held styling device 1 so as to cover on both sides a single treatment surface 2d or 3d, preferably the treatment surface 2d or 3d intended to be heated for the purpose of increasing the temperature of the keratin fibres.

As a further variant, the substrate 6 may wrap at least one of the treatment surfaces 2d or 3d like a food film.

FIG. 2 thus describes the hand-held styling device 1 once the substrates 6 been installed on the treatment surfaces 2d and 3d borne respectively by the jaws 2 and 3.

More specifically, FIG. 2 diagrammatically represents the substrates 6 mounted on the treatment surfaces 2d and 3d in an adjusted manner in order to cover them over the whole of their length. Thus, the substrates 6 totally cover the treatment surfaces 2d and 3d and partially cover the jaws 2 and 3, leaving free a portion of their internal surfaces 2a (respectively 3a) and external surfaces 2b (respectively 3b).

Preferably, the treatment surfaces 2a and 3a are in contact with the substrates 6.

In FIG. 2, the substrates 6 have a closed oblong geometric shape, that is to say they cover not only, on both sides, the treatment surfaces 2d and 3d, but also the free ends of the jaws 2 and 3, in particular the edges 2c and 3c. Preferably, the substrates 6 are elements in sheet form having an oblong shape.

As a variant, the substrates 6 can cover a portion of the free end of the jaws 2 and 3, that is to say the internal surfaces 2a and 3a and also the external surfaces 2b and 3b, while at the same time leaving free the edges 2c and 3c.

As a further variant, the substrates 6 can be sheets having a parallelepipedal shape which cover the treatment surfaces 2d and 3d of the hand-held styling device 1 and a portion of the internal surfaces 2a and 3a of the jaws 2 and 3.

The substrates 6 can be attached on the treatment surfaces 2d and 3d by means of a mechanical or magnetic attachment system, which makes it possible to improve their removable nature and allows them to be rapidly installed and/or removed.

Preferably, the attachment system can be clips or grippers, not represented in FIG. 2.

The substrates can be rapidly installed on and/or removed from the hand-held styling device 1 as a function of the type and number of patterns that it is the aim to produce.

FIG. 3 represents a dispensing means, having a general reference 7, mounted on the external surface 2b of the first jaw 7, comprising two spools 8a and 8b connected to one another by a belt 9, preferably an endless belt, which has an exterior surface 9a. The dispensing means 7 is removably attached to the jaw 2, for example by means of a magnetic system, not represented in FIG. 3.

More specifically, the spool 8a is positioned on the external surface 2b in the vicinity of the edge 2c of the jaw 2 and the spool 8b is positioned on the external surface 2b in the vicinity of the hinge 4

The spools 8a and 8b are mounted so as to rotate freely about a pivoting axis A on the first jaw 2, in accordance with a vertical axis Y-Y′, and operate cooperatively with each other by virtue of the presence of the belt 9 allowing their simultaneous clockwise rotation. The rotational movement of the spools 8a and 8b is actuated manually, mechanically, in a motorized manner or following a movement to close the jaws 2 and 3.

The substrate 6 is mounted on the whole of the dispensing means 7 so as to cover the whole of the first jaw 2, in particular the treatment surface 2d and also the edge 2c. In particular, the substrate 6 is positioned on the spools 8a and 8b and is capable of being driven by the belt 9. During the rotation of the spools 8a and 8b, the substrate 6 moves in a clockwise direction and circulates between the jaws 2 and 3 of the hand-held styling device 1 by virtue of the belt 9. Thus, the dispensing means 7 can correspond to a barrel and makes it possible to circulate the substrate 6 before after each application.

In accordance with this embodiment, the treatment surface 2d is intended to be heated in order to increase the temperature of the keratin fibres.

FIG. 4 represents another embodiment of the dispensing means 7 which is mounted on the external surface 2b of the first jaw 2 and comprises two spools 8c and 8d positioned in the same way as in FIG. 3. The two spools 8c and 8d are removably attached to the jaw 2, for example by means of a magnetic system, not represented in FIG. 4.

The two spools 8c and 8d are connected to one another by means of a belt 10, preferably an endless belt, and are mounted so as to rotate freely about a pivoting axis A on the first jaw 2, said spools being positioned on the external surface 2a in accordance with a vertical axis Y-Y′.

In accordance with this embodiment, the spool 8d represents a spool for feeding the substrate 6 and the spool 8c represents a spool for receiving the substrate 6. By virtue of the presence of the belt 10, the rotation of the spool 8d drives the rotation of the spool 8c.

Initially, the substrate 6 is wound only on the spool 8d, which is positioned on the external surface 2a in the vicinity of the hinge 4. During operation, the spool 8d rotates about its axis of rotation A and the substrate 6 moves, driven by the belt 10, towards the school 8c while passing over the internal surface 2a and the treatment surface 2d of the jaw 2. The substrate 6 is thus placed on the treatment surface 2d when the jaws 2 and 3 are in the open position O before inserting the keratin fibres between said jaws 2 and 3.

The dispensing means 7 can thus correspond to a reel since it makes it possible to circulate the substrate 6 from one spool to another, in the case in point from the spool 8d serving to feed the spool 8c which receives it.

In accordance with this embodiment, the treatment surface 2d is intended to be heated in order to increase the temperature of the keratin fibres.

In accordance with FIGS. 3 and 4, the dispensing means 7 thus represented has the advantage of producing several identical or different coloured or bleached patterns while at the same time dispensing with the regular operations of installing and removing the substrate(s).

The use of such a dispensing means proves to be particularly advantageous for producing varied and/or numerous patterns while at the same time changing substrates as little as possible.

In accordance with the embodiments of FIGS. 3 and 4, the substrate can advantageously correspond to a ribbon comprising, on one and the same surface, several layers of different geometric shapes capable of producing varied coloured or bleached patterns. Thus, the dispensing means 7 makes it possible to use the various layers of one and the same substrate in the form of a ribbon.

FIG. 5 diagrammatically represents an assembly, having the general reference 11, comprising the hand-held styling device 1 as described in FIG. 2, that is to say the free ends of the jaws 2 and 3 of which are covered with the substrates 6, a computing device 12 and an image acquisition device 13.

In FIG. 5, the first jaw 2 further comprises detection means 14 positioned on the internal surface 2a and the second jaw 3 further comprises work detection means 15 positioned on the internal surface 3a.

The detection means 14 and 15 are capable of detecting the movement of the jaws 2 and 3 into the open position O and into the closed position F. The detection means 14 and 15 are respectively connected to the computing device 12 by means of circuits 16 and 17.

Thus, the detection means 14 and 15 make it possible to provide information on the number of times that one and the same substrate has been used to produce one or more coloured or bleached patterns and/or the number of times that one and the same substrate can still be used before total extraction of the dyes.

In accordance with the embodiment described in FIG. 5, the computing device 12 is connected to an image acquisition device 13 by means of a circuit 18 which makes it possible to visualize, on the screen, the type and number of coloured or bleached patterns produced on the keratin fibres.

In addition, the first jaw 2 can comprise heating control means 19 intended to control the temperature of the treatment surface 2d of the hand-held styling device 1. According to FIG. 5, the heating control means 19 are connected to the computing device 12, in particular to a control member, which is not represented, by means of a circuit 20.

Thus, after the application of the substrate 6, the control member of the computing device 12 can reduce the temperature of the treatment surface 2d, in particular to a temperature of less than 60° C., in particular less than 30° C., so as to protect the integrity of the dyes and/or of the oxidizing agents against heat and to prolong the lifetime of the substrate 6.

In particular, it is possible to provide information about a determined target temperature below which the temperature of the treatment surface 2d must be maintained.

The control member of the computing device 12 also proves to be particularly advantageous when it is a question of producing coloured or bleached patterns on different models in the same period of time using various hand-held styling devices 1. Indeed, after the production of coloured or bleached patterns on a portion of the head of hair of one model, the temperature of the treatment surface 2d of the hand-held styling device 1 can be decreased and the stylist can use another hand-held styling device in order to produce a colouring on another model. In this way, the stylist can then continue to produce the coloured or bleached patterns of the previous model without having to change substrate.

FIG. 6 diagrammatically represents an assembly, having the general reference 21, comprising the hand-held styling device 1, as described in FIG. 4, that is to say equipped with a dispensing device 7 comprising the spools 8c and 8d, the computing device 12 and the image acquisition device 13.

In this embodiment, the computing device 12 is directly connected to the dispensing device 7, in particular to the receiving spool 8c, in particular by means of circuits 22 and 23.

The combined presence of the computing device 12 and of the image acquisition device 13 makes it possible in addition to help the user identify the pattern(s) (or the layers containing the dyes and/or the chemical oxidizing agents) that are in place in order to perform the dyeing or the lightening. Quite naturally, the two devices 12 and 13 also make it possible to calculate the number of uses of coloured or bleached patterns produced and the number that remain to be produced.

In accordance with this embodiment, the computing device 12 may comprise a motor card making it possible to order the dispensing means 7 to move the substrate 6 in order to place the desired pattern before performing the dyeing or the lightening. The controlling of the dispensing means 7 by the computing device 12 is carried out by means of the circuits 22 and 23.

FIG. 7 diagrammatically represents an assembly, having the general reference 24, comprising the hand-held styling device 1, as described in FIG. 4, that is to say equipped with a dispensing device 7 comprising the spools 8c and 8d, the computing device 12 and the image acquisition device 13.

In accordance with FIG. 7, the first jaw 2 comprises one or more closure means 25, preferably a closure sensor, for closing the jaws 2 and 3 and connected to the computing device 12 by means of a circuit 26.

The closure means 25 can be used before actuating the dispensing means 7, in particular the spool 8d, making it possible to place another substrate or another layer of the same substrate.

The use of the closure means 25 is controlled by the computing device 12.

Thus, the assembly described in FIG. 7 is a semi-automatic system since the jaws 2 and 3 can be in the closed position by virtue of the control of the computing device 12.

FIG. 8 diagrammatically represents an assembly, having the general reference 26, comprising the hand-held styling device 1, as described in FIG. 4, that is to say equipped with a dispensing device 7 comprising the spools 8c and 8d, the computing device 12 and the image acquisition device 13.

In accordance with FIG. 8, the first jaw 2 and the second jaw 3 comprise a closure means 27 for closing the jaws 2 and 3 and an opening means 28 for opening the jaws 2 and 3. Preferably, the closure means 27 is an electromagnet and the opening means 28 is a return spring. In other words, the closure means 27 ensures the actuation of a closed position of the jaws 2 and 3, whereas the opening means 28 ensures the actuation of a closed position of the jaws 2 and 3.

The closure means 27 and the opening means 28 are connected to the computing device 12 by means of a circuit 29.

Thus, the assembly described in FIG. 8 is an automatic system since the jaws 2 and 3 can be in the closed position and open position by virtue of the control of the computing device 12.

The computing device 12 may comprise a piece of software 30 configured for recording and loading the dyeing or lightening patterns that it is the aim to produce on the keratin fibres, in particular at various places on the head of hair.

By virtue of this embodiment, the user can record and load, optionally remotely, in the hair salon of their choice, the type of coloured or bleached patterns, in particular their geometric shape or their colour, and also the number of coloured patterns that they wish to produce on a head of hair.

In accordance with these various embodiments, the substrate(s) used comprise a surface coated with at least one layer containing one or more dyes, preferably chosen from direct dyes and/or oxidation dyes, more preferably oxidation dyes.

Preferably, the oxidation dyes are chosen from heterocyclic bases and heterocyclic or benzene-based couplers with at least one amine substituent on the aromatic ring.

Preferably, the oxidation dyes are chosen from couplers and more preferably heterocyclic or benzene-based couplers with at least one amine substituent on the aromatic ring.

As a variant, the substrate(s) used comprise a surface coated with at least one layer containing one or more oxidizing agents.

### EXAMPLE 1: DYEING ON NATURAL HAIR

A solution A of sodium persulfate at 10 g % in water is prepared (spontaneous pH=3.4±0.2).

The dyeing composition B is prepared:

TABLE 6

| | B |
|---|---|
| | (g %) |
| DENATURED ABSOLUTE ETHYL ALCOHOL | 8.8 |
| PROPYLENE GLYCOL | 6.2 |
| HEXYLENE GLYCOL | 3 |
| DIPROPYLENE GLYCOL | 3 |
| (C₁₄/₁₆) ALKYL | 2 |
| HYDROXYETHYLCELLULOSE | |
| ASCORBIC ACID | 0.25 |
| POWDERED SODIUM METABISULFITE | 0.25 |
| PURE MONOETHANOLAMINE | 2 |
| 4-(3-AMINOPYRAZOLO[1,5-A]PYRIDIN-2-YL)-1,1- | 1.83 |
| DIMETHYLPIPERAZIN-1-IUM CHLORIDE | |
| HYDROCHLORIDE | |
| 1-METHYL-2-HYDROXY-4-BETA- | 0.96 |
| HYDROXYETHYL AMINOBENZENE | |
| DEIONIZED WATER | qs 100 |

The pH of composition B is 9.58±0.2.

A lock of natural hair is then dyed according to the following protocol:
Solution A is applied to a lock of Caucasian hair comprising 90% grey hairs for 10 min (solution/hair bath ratio: 10/1 by weight).
After this leave-on time, the lock is rinsed.
Using a brush, 10 g of composition B are distributed on Kimtech 7505 adsorbent paper from Kimberly-Clark (dimensions: 10 cm×20 cm).
The paper is then placed between the tongs of a straightening iron, regulated at 180° C.
The lock is sandwiched in the paper, then the tongs of the straightening iron are pressed together for 10 seconds.
The lock is rinsed, washed with Ultra doux camomille shampoo [Ultra soft shampoo with chamomile] and dried.
The colorimetric values are measured using a Konica Minolta CM-3600A spectrocolorimeter.

A strong purple colouration is obtained.

TABLE 7

| | L* | a* | b* | Δₐ*_{b}* |
|---|---|---|---|---|
| Natural Caucasian lock comprising 90% grey | 62.28 | 1.25 | 17.12 | |
| hairs before treatment | | | | |
| Natural Caucasian lock comprising 90% grey | 32.31 | 9.33 | −9.03 | 40.58 |
| hairs after treatment | | | | |

### EXAMPLE 2: DYEING ON BLEACHED HAIR

The following compositions are prepared:

Dyeing Composition C

TABLE 8

| | C (g %) |
|---|---|
| DENATURED ABSOLUTE ETHYL ALCOHOL | 8.8 |
| PROPYLENE GLYCOL | 6.2 |
| HEXYLENE GLYCOL | 3 |
| DIPROPYLENE GLYCOL | 3 |
| (C₁₄/₁₆) ALKYL HYDROXYETHYLCELLULOSE | 2 |
| ASCORBIC ACID | 0.25 |
| POWDERED SODIUM METABISULFITE | 0.25 |
| PURE MONOETHANOLAMINE | 2 |
| 2,3-DIAMINO-6,7-DIHYDRO-1H,5H-PYRAZOLO[1,2- | 2 |
| A]PYRAZOL-1-ONE DIMETHANESULFONATE | |
| 1-BETA-HYDROXYETHYLOXY-2,4-DIAMINOBENZENE | 1.43 |
| DICHLORHYDRATE | |
| DEIONIZED WATER | qs 100 |

Bleaching Powder D

TABLE 9

| | D |
|---|---|
| POTASSIUM PERSULFATE | 32.9 |
| AMMONIUM PERSULFATE | 9.8 |
| SODIUM SILICATE HYDRATE | 33.7 |
| MAGNESIUM CARBONATE PENTAHYDRATE | 9.2 |
| ETHYLENEDIAMINETETRACETIC ACID DIHYDRATE, | 1 |
| DISODIUM SALT | |
| ACRYLIC ACID/STEARYL METHACRYLATE COPOLYMER | 0.7 |
| POLYMERIZED IN AN ETHYL ACETATE/CYCLOHEXANE | |
| MIXTURE | |
| HYDROXYETHYLCELLULOSE (MW: 1 000 000) | 0.7 |
| L-GLYCINE | 1 |
| TALC | 6.85 |
| ULTRAMARINE BLUE (CI: 77007) | 0.25 |
| POLYDIMETHYLSILOXANE (VISCOSITY: 5 CST) | 1.4 |
| WHITE MINERAL OIL | 2.5 |

Oxidizing Composition E

TABLE 10

| | E |
|---|---|
| CETYLSTEARYL ALCOHOL/OXYETHYLENATED | 2.85 |
| CETYLSTEARYL ALCOHOL (30 OE) MIXTURE | |
| (50% LINEAR 70/30 C13/C15) ALKYL ETHER | 0.85 |
| CARBOXYLIC ACID MONOETHANOLAMIDE (2 OE) | |
| GLYCEROL | 0.5 |
| TETRASODIUM PYROPHOSPHATE DECAHYDRATE | 0.04 |
| SODIUM SALICYLATE | 0.035 |
| ETIDRONIC ACID, TETRASODIUM SALT, AS AN | 0.2 |
| AQUEOUS 30% SOLUTION | |
| HYDROGEN PEROXYDE AS A 50% SOLUTION (200 | 23.5 |
| VOL. AQUEOUS HYDROGEN PEROXIDE SOLUTION) | |
| MICROBIOLOGICALLY CLEAN DEIONIZED WATER | qs 100 |

The pH of composition C is 9±0.2.

A lock of natural hair is then dyed according to the following protocol:
A bleaching product is prepared by mixing 1 part by weight of bleaching powder D with 2 parts by weight of an oxidizing composition E.
The mixture is applied to a lock of TD8 chestnut-brown Caucasian hair for 50 min (solution/hair bath ratio: 10/1 by weight).
After this leave-on time, the lock is rinsed.
Using a brush, 10 g of composition C are distributed on Kimtech 7505 adsorbent paper from Kimberly-Clark (dimensions: 10 cm×20 cm).
The paper is then placed between the tongs of a straightening iron, regulated at 180° C.
The lock is sandwiched in the paper, then the tongs of the straightening iron are pressed together for 10 seconds.
The lock is rinsed, washed with Ultra doux camomille shampoo [Ultra soft shampoo with chamomile] and dried.

The colorimetric values are measured using a Konica Minolta CM-3600A spectrocolorimeter.

A strong red colouration is obtained.

TABLE 11

| | L* | a* | b* | Δₐ*_{b}* |
|---|---|---|---|---|
| Bleached TD8 Caucasian lock before | 43.05 | 8.28 | 21.09 | |
| treatment | | | | |
| Bleached TD8 Caucasian lock after | 44.1 | 31.74 | 24.32 | 23.7 |
| treatment | | | | |

### EXAMPLE 3: LIGHTENING OF A NATURAL HAIR

A bleaching solution is prepared: 10 g of bleaching powder D are diluted in water (spontaneous pH=11).

A lock of natural TD4 Caucasian hair is then lightened according to the following protocol:
The bleaching solution is applied to the lock of hair for 10 min (solution/hair bath ratio: 10/1 by weight).
After this leave-on time, the lock is rinsed.
Using a brush, 10 g of oxidizing composition E are distributed on Kimtech 7505 adsorbent paper from Kimberly-Clark (dimensions: 10 cm×20 cm).
The paper is then placed between the tongs of a straightening iron, regulated at 180° C.
The lock is sandwiched in the paper, then the tongs of the straightening iron are pressed together 5 times (time of each passage: 10 seconds).
The lock is rinsed, washed with Ultra doux camomille shampoo [Ultra soft shampoo with chamomile] and dried.

The colorimetric values are measured using a Konica Minolta CM-3600A spectrocolorimeter.

A lightening is obtained.

TABLE 12

| | L* | a* | b* | Δₐ*_{b}* |
|---|---|---|---|---|
| TD4 Caucasian lock before treatment | 21.85 | 3.31 | 4.35 | |
| TD4 Caucasian lock after treatment | 24.33 | 5.46 | 7.38 | 6.5 |

1. Process for dyeing and/or lightening keratin fibres, in particular human keratin fibres such as the hair, using, on said fibres, at least one aqueous composition and at least one hand-held styling device (1) comprising:
a first and a second jaw (2, 3) arranged facing one another and connected to one another so as to go from an open position (O) to a closed position (F),
a first treatment surface (2d), borne by the first jaw (2), and a second treatment surface (3d), borne by the second jaw (3), the treatment surfaces (2d, 3d) being intended to grip said keratin fibres, and at least one of the treatment surfaces (2d, 3d) being intended to be heated,
at least one of the treatment surfaces (2d, 3d) being partially or totally covered with at least one substrate (6) comprising a surface (6a) coated with at least one layer (6b) containing one or more dyes, preferably chosen from oxidation dyes and/or direct dyes, and/or one or more chemical oxidizing agents.

2. Process according to claim 1, characterized in that the oxidation dyes are chosen from couplers and oxidation bases.

3. Process according to claim 1, characterized in that the direct dyes are chosen from neutral and/or cationic and/or anionic direct dyes.

4. Process according to claim 1, characterized in that the substrate (6) is an element in sheet form.

5. Process according to claim 1, characterized in that the substrate (6) is mounted totally or partially mobile on at least one of the treatment surfaces (2d, 3d) of the hand-held styling device (1).

6. Process according to claim 5, characterized in that the substrate (6) is oblong in shape and covers, on both sides, at least one of the treatment surfaces (2d, 3d) of the hand-held styling device (1).

7. Process according to claim 1, characterized in that the substrate (6) is mounted totally or partially mobile on at least one of the treatment surfaces (2d, 3d) of the hand-held styling device (1) by means of a mechanical attachment system comprising one or more mechanical attachment means.

8. Process according to claim 1, characterized in that the substrate (6) is mounted mobile on at least one of the treatment surfaces (2d, 3d) of the hand-held styling device (1) by means of at least one dispensing means (7), which pivots about a pivoting axis (A), positioned on the first jaw (2) or second jaw (3), in accordance with a vertical axis Y-Y′, and is capable of being driven manually, mechanically, in a motorized manner or following a jaw (2, 3) closure movement.

9. Process according to claim 8, characterized in that the dispensing means (7) is a reel or a barrel.

10. Process according to claim 1, characterized in that the first and/or the second jaw (2, 3) of the hand-held styling device (1) comprises one or more means (14, 15) for detecting the moving into the open position (O) and/or the moving into the closed position of the jaws (2, 3) and connected to a computing device (12) configured for calculating the number of uses of the substrate(s) (6) to the keratin fibres.

11. Process according to claim 1, characterized in that the first and/or the second jaw (2, 3) of the hand-held styling device (1) comprises one or more means (25) for closing the jaws (2, 3).

12. Process according to claim 1, characterized in that the aqueous composition is a composition comprising one or more chemical oxidizing agents, preferably chosen from hydrogen peroxide and/or peroxygenated salts.

13. Process according to claim 1, characterized in that it comprises:
using an aqueous composition, as defined on said fibres,
applying the treatment surfaces of the hand-held styling device to said keratin fibres,
increasing the temperature of said fibres to a temperature ranging from 60 to 250° C.

14. Assembly (11, 24, 26) intended for dyeing and/or lightening keratin fibres, in particular human keratin fibres such as the hair, characterized in that it comprises at least one aqueous composition and at least one hand-held styling device (1) as defined according to claim 1.

15. Assembly (11, 24, 26) according to claim 14, characterized in that it comprises a computing device (12) configured for calculating the number of uses of the substrate(s) (6) on the keratin fibres and a styling device, in which the first and/or the second jaw (2, 3) also comprises one or more means (14, 15) for detecting the moving into the open position (O) and/or the moving into the closed position of the jaws (2, 3); said means (14, 15) being connected to the computing device (12).

16. Assembly according to claim 15, characterized in that the computing device (12) is connected to an image acquisition device configured for visualizing on a screen the surface of the substrate(s) (6) used on the keratin fibres.

17. Assembly according to claim 15, characterized in that the computing device (12) comprises a control member configured for decreasing the heating temperature of the treatment surface (2d, 3d) intended to be heated of the hand-held styling device (1).

18. Assembly according to claim 15, characterized in that the computing device (12) comprises at least one piece of software configured for recording and charging the dyeing or lightening patterns that it is the aim to produce on the keratin fibres.
